(19) United States
(12) Patent Application Publication
(10) Pub. No.: US 2018/0303974 A1
(43) Pub. Date: Oct. 25, 2018

(54) COMPOSITIONS FOR REGENERATION AND REPAIR OF NEURAL TISSUE

(71) Applicant: Ariel-University Research and Development Company Ltd., Ariel (IL)

(72) Inventors: Danny BARANES, Beer-Sheva (IL); Orly Eva WEISS, Petach-Tikva (IL); Eyal CHANJI, Yavne (IL); LIat HAMMER, ModiIn (IL); Tzachy MORAD, Moshav Beit Yitzhak (IL); Ido MERFIELD, Nirit (IL)

(73) Assignee: Ariel-University Research and Development Company Ltd., Ariel (IL)

(21) Appl. No.: 15/769,349

(22) PCT Filed: Nov. 3, 2016
(86) PCT No.: PCT/IL2016/051193

§ 371 Date: Apr. 19, 2018

Related U.S. Application Data
(60) Provisional application No. 62/250,017, filed on Nov. 3, 2015.

(51) Int. Cl. A61L 27/46; A61L 27/44; A61L 27/38; A61L 27/36; A61L 27/54

(52) U.S. Cl. CPC A61L 27/46; A61L 27/446; A61L 27/38; A61L 27/3616; A61L 2300/43; A61L 27/54; A61L 2430/32; A61L 2300/414; A61L 2300/426; A61L 27/3675

(57) ABSTRACT

Methods and compositions for repair and regeneration of neural tissue are provided. Particularly, methods and compositions for promoting neural tissue wound healing and treatment of traumatic brain injury using porous crystalline calcium carbonate particles and a biocompatible polymer, for example compositions comprising porous coral exoskeleton particles in combination with a biocompatible polymer, and optionally comprising neural growth agents and platelets for application to damaged neural tissue for enhancing neural regrowth and recovered functionality, in, for example, but not limited to, traumatic brain injury (TBI).

### FIELD AND BACKGROUND OF THE INVENTION

The present invention, in some embodiments thereof, relates to neural tissue regeneration and repair and, more particularly, but not exclusively, to compositions and methods for treatment of traumatic CNS injury.

Traumatic brain injury (TBI), spinal cord injuries and other neurological disorders such as Alzheimer and stroke have major clinical and social impact on humanity. TBI is the leading cause of death in young adults in the United States and a major cause of death and disability worldwide. Each year approximately 1.5 million head injuries are treated in United States and Europe, with approximately 50,000 deaths occurring from TBI in the US alone. According to US Centers for Disease Control and Prevention, most TBI cases are related to falls and sport injuries, with vehicle accidents and other impacts accounting for approximately 30%. The economic cost of TBI in the United States alone in 2010 was estimated to be approximately $76.5 billion.

TBI causes destruction, necrosis and phagocytosis of brain tissue, resulting in large cortical defects with respective functional deficits, which, due to their large size and formation of astrocytic scar at the walls of the wound cannot be regenerated by intrinsic repair mechanisms, (i.e. neurogenesis). Such large cortical defects can also result in distant changes in the brain, (e.g. long-term neurodegeneration). In fact, it has been known for some time that moderate and severe head injury are associated with increased risk of Alzheimer's disease in later life.

In the adult central nervous system (especially that of elderly people, where intervention is mostly needed) there is a paucity of appropriate endogenous factors and cellular machinery for damage recovery. As a consequence, injury or disease causes severe neural cell loss and insufficient tissue restoration. To date, the need for effective TBI and spinal cord injury treatment has gone largely unmet, with most practice guidelines offering rest, rehabilitation, and gradual return to activities as the cardinal touchstones. The lack of tools for direct biological intervention is striking both on an economic and a human level.

While many factors influencing neural cell differentiation, growth and maturation have been identified, none have succeeded in providing effective remedies for traumatic brain injury.

Bio-compatible, three-dimensional scaffolds have been proposed for neural tissue repair and regeneration. Ideally, such a scaffold would be capable of eliminating inflammation, minimizing progression of injury, stimulating growth and differentiation of nerve progenitors, delivering cells to replace cells and tissue lost to injury and promoting cell viability and proliferation-able to resist structural collapse while supporting the endogenous restorative mechanisms of the injured brain and the slow and complex processes of neurovascularization, neurogenesis, and neural reorganization. However, tissue engineering strategies have largely failed to address the need for enhanced generation and migration of neurons to the damaged sites in the nervous system, and promotion of their synaptic interaction. A variety of natural and synthetic materials have been studied as candidate scaffolds, mostly hydrogels, in an attempt to recreate the natural tissue environment (see, for example, US Patent Application 20050226856 to Ahlfors).

Implantation of hydrogel-based scaffolds attempted to supply the cells near the wound with mechanical support and a growth matrix so that they could migrate into the injury site for tissue restoration. Hydrogel scaffolds were produced synthetically or generated from biological sources, leading to degradable synthetic polymers such as poly (alpha-hydroxyacids), polylactic acid, polyglycolic acid, poly (lactic-co-glycolic acid) (PLGA), poly (L-lactic acid)(PLLA) and the like. Such polymers have the advantage of being easily machined and formed into tailored shapes with sufficient mechanical strength, however, they have failed to generate cell-recognition signals, resulting in insufficient cell adhesion and hydrophobicity. Biomaterial gels such as of the protein collagen, although useful as a substrate for neurons in culture and in vivo, were found to be mechanically unstable and tended to collapse too soon after implantation, leading to nerve compression and failure of the implant.

Coral skeleton (coral, coralline, aragonite, hydroxyapatite, etc) is microporous and possesses a high ratio of surface area per volume. Coral scaffolds have been used to support ex-vivo growth of cells (see, for example, US Patent Application 2005/0053585 to Black et al.; for review see Vago, Organogenesis 2008 4:18-22), and for implantation into tissue, predominantly for musculoskeletal (bone, cartilage, etc) tissue repair (see, for example, US Patent Application No. 20110256228 to Altschuler). Coral skeleton scaffolds have also been used for growth of nervous system-derived cells (astrocytes, neurons) and formation of ganglion-like structures in vitro on coral skeleton scaffolds (Shany et al., Tissue Engineering 2006; 12:1-11; Peretz et al. Tissue Engineering 2007; 13:461-72; Baranes et al. Tissue Engineering 2007; 13:473-482).

Other relevant publications include US Patent Application No. 20140294913 to Hasirci, et al.; US Patent Application No. 20040005297 to Connelly et al. and US Patent Application No. 20130226310 to Schwartz et al.

### SUMMARY OF THE INVENTION

According to an aspect of some embodiments of the present invention there is provided a method for regenerating or repair of neural tissue comprising contacting the neural tissue with a composition comprising porous crystalline calcium carbonate or calcium phosphate particles and a biocompatible polymer.

According to an aspect of some embodiments of the present invention there is provided a composition for regenerating or repair of neural tissue comprising porous crystalline calcium carbonate or calcium phosphate particles, a biocompatible polymer and a nerve growth or regeneration agent.

According to some embodiments of the present invention the neural tissue comprises a tissue selected from the group consisting of neuronal tissue, glial tissue and central nervous system neural tissue.

According to some embodiments of the present invention the neural tissue comprises central nervous system tissue.

According to some embodiments of the present invention the neural tissue comprises brain tissue.

According to some embodiments of the present invention the method further comprising contacting the neural tissue with a nerve growth or regeneration agent.

According to some embodiments of the present invention the nerve growth or regeneration agent is selected from the group consisting of insulin, epidermal growth factor (EGF), brain derived neurotrophic factor (BDNF), glial cell-derived neurotrophic factor (GDNF), nerve growth factor (NGF), VGF nerve growth factor (VGF), neurotropin-3 (NT3) and cytokines.

According to some embodiments of the present invention the composition comprises the nerve growth or regeneration agent.

According to some embodiments of the present invention the contacting neural tissue is effected in vivo in a subject in need thereof.

According to some embodiments of the present invention the subject is a human subject.

According to some embodiments of the present invention the neural tissue is injured or damaged neural tissue.

According to some embodiments of the present invention the injury or damage is caused by condition selected from the group consisting of peripheral nerve injury or neuropathy, cranial or cerebral trauma, aneurysm, spinal cord injury, stroke and disease.

According to some embodiments of the present invention the disease is selected from the group consisting of Alzheimer's disease, Parkinson's disease, Huntington's disease, multiple sclerosis, Creutzfeldt-Jacob disease, kuru, multiple system atrophy, amyotropic lateral sclerosis (Lou Gehrig's disease), progressive supranuclear palsy, optic neuritis, diabetic retinopathy, macular degeneration and glaucoma.

According to some embodiments of the present invention the composition comprises cells.

According to some embodiments of the present invention the cells are seeded upon the composition optionally prior to the contacting.

According to some embodiments of the present invention the nerve growth or regeneration agent is selected from the group consisting of insulin, epidermal growth factor (EGF), brain derived neurotrophic factor (BDNF), glial cell-derived neurotrophic factor (GDNF), nerve growth factor (NGF), VGF nerve growth factor (VGF), neurotropin-3 (NT3) and cytokines.

According to some embodiments of the present invention the biocompatible polymer is biodegradable.

According to some embodiments of the present invention the composition is biodegradable.

According to some embodiments of the present invention the porous calcium phosphate particles comprise hydroxyapatite particles.

According to some embodiments of the present invention the porous crystalline calcium carbonate is selected from the group consisting of aragonite, calcite, ikaite, vaterite and monohydrocalcite.

According to some embodiments of the present invention the porous crystalline calcium carbonate comprises aragonite.

According to some embodiments of the present invention the porous crystalline calcium carbonate comprises acellular coral exoskeleton.

According to some embodiments of the present invention the acellular coral exoskeleton comprises coral exoskeleton from coral of the Porites species.

According to some embodiments of the present invention the coral exoskeleton comprises Portia lutea coral exoskeleton.

According to some embodiments of the present invention the crystalline calcium carbonate particles have an average particle diameter of between about 1 micrometer and about 5 mm.

According to some embodiments of the present invention the polymer comprises a natural polymer.

According to some embodiments of the present invention the natural polymer is selected from the group consisting of collagen, albumin, fibrinogen, elastin, silk, hyaluronic acid and chitosan.

According to some embodiments of the present invention the polymer comprises collagen.

According to some embodiments of the present invention the biocompatible polymer is uniform in density.

According to some embodiments of the present invention the composition comprises biocompatible polymers of a plurality of densities.

According to some embodiments of the present invention the polymer comprises 2-6 mg/ml collagen.

According to some embodiments of the present invention the porous crystalline calcium carbonate particles are distributed evenly within the biocompatible polymer.

According to some embodiments of the present invention the porous crystalline calcium carbonate particles are unevenly distributed within the biocompatible polymer.

According to some embodiments of the present invention the porous crystalline calcium carbonate particles distributed on the external surface thereof.

According to some embodiments of the present invention the composition comprises the porous crystalline calcium carbonate particles and the biocompatible polymer in a ratio of at least 33 μg calcium carbonate particles per milliliter biocompatible polymer.

According to some embodiments of the present invention the composition comprises insulin and/or EGF.

According to some embodiments of the present invention the composition comprises insulin and/or platelets.

According to some embodiments of the present invention the platelets comprise platelet-rich-plasma.

According to some embodiments of the present invention the composition comprises cells.

According to an aspect of some embodiments of the present invention there is provided a pharmaceutical composition comprising any one of the compositions of the invention and a pharmaceutically acceptable carrier.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A and 1B are photographs of particles of Porites lutea coral skeleton following graining to millimeter size (FIG. 1A) or sub-millimeter size (FIG. 1B);

FIGS. 2A-2D show images of brains of adult mice, one week after implantation of coral skeleton particles or glass beads (0.1 mm diameter) (control) into the frontal cortex. FIG. 2A shows the whole brain and sites of implantation. FIG. 2B shows the sites of implantation in a sagittal section of the brain. Toluidine blue staining, labeling all cells, revealed high color absorbance surrounding the coral skeleton particle implant, and little staining with the glass beads implants (FIG. 2C). Coral skeleton particles remain adherent following extensive wash (FIG. 2D), while glass beads are easily washed out;

FIGS. 3A-3C are images of immunofluorescent staining showing partial repair of cortical injury in adult brain following implantation of coral skeleton particles. FIG. 3A (upper panel, arrows) shows implanted coral skeleton particles in the cortex, and adherent cells (lower panel-nuclei, DAPI staining in red). FIG. 3B shows GluR1 expressing neurons, (arrows), growing on the coral skeleton implant. FIG. 3C shows the formation of an astrocytic bridge (red, GFAP, blue-cell nuclei, DAPI, right arrow) in the cortical wound (left arrow) in the vicinity of the implanted coral skeleton particles (FIG. 3C). Scale of 3B and 3C is 60 μm;

FIG. 4 is a graphic representation of two types of implants used: a coral skeleton-collagen (CC) implant and a coral skeleton-collagen+platelet-rich plasma+insulin (CCPI) implant, indicating some advantages of the implant material;

FIGS. 5A-5E are photographs illustrating the coral skeleton-collagen implant, showing its form (FIG. 5A), pliability (FIGS. 5B and 5C), and characteristics of an exemplary collagen outer layer encapsulating the dense inner coral layer comprising coral skeleton particles (FIGS. 5D and 5E);

FIG. 6 is a photograph illustrating recovery of nervous tissue at an adult mouse brain cortical wound site following implantation of a coral skeleton-collagen implant. Synapse-specific staining of sections of the wound site identified accumulation of cells (nuclei(DAPI), blue) and pre-synaptic termini (synaptic vesicle 2 (SV2), green) at the wound site, with sparse expression of post-synaptic markers (glutamate receptor 1 (GluR1), red);

FIGS. 7A-7D are photographs illustrating the recovery of a cortical wound in adult mice, following implantation of a coral skeleton-collagen implant together with insulin and EGF, one month after implantation. FIG. 7A shows the implanted region following insertion of the implant, and FIG. 7B is a composite of confocal sections illustrating the presence of coral skeleton particles within the wound. FIG. 7C is a photo of a 50 μm confocal section through the wound site showing accumulation of cells (nuclei, blue (DAPI)) within the wound and growth of new nervous tissue containing neurons with pre-synaptic termini (SV2, green) and post-synaptic clusters (GluR1, red) (FIG. 7D);

FIGS. 8A and 8B are photographs illustrating the recovery of a cortical wound in adult mice, following implantation of a coral skeleton-collagen-platelets implant. FIG. 8A shows the implant in-situ in the cortex. FIG. 8B is a confocal section through the cortical wound two weeks after implantation, showing neural regeneration (astrocytes (GFAP) stain red, neuroblasts (nestin) stain green) amid the regrowth of cells (cell nuclei stain blue (DAPI)) forming a continuous mass across the injured region of the cortex. Note that most of the wound border lacks scar tissue, and the high density of neuroblasts (green) within the recovering wound, exceeding that of the surrounding cortical tissue;

FIG. 9 is a graph of the weights of implanted and control adult mice, 1-12 days post-cortical injury. Legend: Blue-no injury (control); Orange-cortical injury (as described); Green-injury+implants of coral skeleton particles; Purple-injury+implant of coral skeleton with collagen. No significant or out of the ordinary weight perturbation was recorded in the implanted or control mice;

FIGS. 10A-10B are phase contrast images illustrating the stabilizing effect of coral skeleton-collagen implants on cortical wound in adult mice. FIGS. 10A and 10B are phase contrast images of sections of the mouse brains, two weeks following injury, showing the contours of the wound in mice brains receiving the coral skeleton-collagen implants (FIG. 10B), compared to the wounds in mice brains without implants (FIG. 10A). Contours of the wound walls are traced below the images—10A—No implant-Collapsed wound-blue and red lines indicate lower and upper wound surfaces, respectively. White arrow is wound's void volume, red arrow indicates location of wound surface overlap. 10B—Implanted wound-no collapse—red line indicates intact wound surfaces. Blue arrows-tight implant-tissue contact sites;

FIGS. 11A-11B are fluorescent images illustrating enhanced cellular replenishment into cortical wounds in adult mice receiving coral skeleton-collagen implants (FIG. 11 B), compared to non-implanted wounds (FIG. 11A), one month following injury. Presence of cells migrating into the wound border (yellow line) is evident in the implanted wound (FIG. 11B, red arrow), but absent in the non-implanted wound (FIG. 11B).

FIG. 11C is a histogram showing quantification of cell density within the site of a cortical wound in implanted (“CC”, “CCPI”) and non-implanted (“Injury”) mouse brains one month following the injury, further illustrating the significant cellular replenishment following implantation of coral skeleton-collagen and coral skeleton-collagen-platelet-rich-plasma+insulin implants;

FIGS. 12A-12B are fluorescent images illustrating migration of neuroblasts into cortical wounds implanted with coral skeleton-collagen-platelet-rich-plasma+insulin implants. Brain slices were stained for DAPI (blue in FIG. 12A, red in FIG. 12B) and nestin (green)-bottom region of image, close to the implant (red arrows, FIG. 12A) shows high density of nestin expressing cells. FIG. 12B is a magnification of the outlined area of FIG. 12A, showing the border of the wound (yellow line) and neuroblasts (red DAPI stain) migrating towards the implant. Scale—A-50 μm, B-12 μm;

FIGS. 13A-13C are photographs illustrating increased neuronal content of tissues around cortical wounds implanted with coral skeleton-collagen-platelet-rich-plasma+insulin implants. FIG. 13A is a photograph of a whole brain one month after cortical injury, showing more extensive repair with coral skeleton-collagen-platelet-rich-plasma+insulin implants (CCPI) compared with coral skeleton-collagen (CC) implants. Immunofluorescent staining for neurons (MAP2-green; NFM-red; DAPI-blue) shows significant neurite (axons and dendrites) density in implanted wounds (FIG. 13C), with greater density around coral skeleton-collagen-platelet-rich-plasma+insulin implants (blue border) than around coral skeleton-collagen implants (red border). Red arrow indicates implant site. Non-implanted wounds (FIG. 13B) exhibited no neuronal accumulation. Scale—B-50 μm, C-200 μm;

FIGS. 14A-14C are histograms illustrating enhanced recovery of functional deficits in traumatic brain injured mice treated with coral skeleton-collagen-platelet-rich-plasma+insulin implants. Brain-injured mice receiving CCPI implants (black columns) showed improved open field test performance (5 minutes per examination): increased walking distance and velocity, FIG. 14A; reduced anxiety (center entries), FIG. 14B, and increased curiosity (rearing episodes), FIG. 14C with the CCPR implants, compared to non-implanted controls.

### DESCRIPTION OF SPECIFIC EMBODIMENTS OF THE INVENTION

The present invention, in some embodiments thereof, relates to methods and compositions for repair and regeneration of neural tissue and, more particularly, but not exclusively, methods and compositions using porous crystalline calcium carbonate particles and a biocompatible polymer for promoting neural tissue wound healing and treatment of traumatic brain injury. In particular, the present invention discloses compositions comprising porous coral exoskeleton particles in combination with a biocompatible polymer, and optionally comprising neural growth agents for application to damaged neural tissue for enhancing neural regrowth and recovered functionality, in, for example, but not limited to, traumatic brain injury (TBI).

Before explaining at least one embodiment of the disclosure in detail, it is to be understood that the disclosure is not necessarily limited in its application to the details set forth in the following description or exemplified by the Examples. The disclosure is capable of other embodiments or of being practiced or carried out in various ways. Also, it is to be understood that the phraseology and terminology employed herein is for the purpose of description and should not be regarded as limiting.

Due to the absence of axonal regeneration, loss of structural integrity and astrocytic scarring, nervous system injury, or disease, often results in large, cystic cortical defects with respective functional deficits, long-term neurodegeneration and increased risk of neurological disease later in life. Materials that can accelerate generation and migration of neurons to the damaged site and cause them to synaptically interact are lacking, and the three dimensional scaffolds commonly employed for tissue engineering (e.g. hydrogels) have disappointingly proven ineffective for neural tissue repair and regeneration.

The present inventors have surprisingly shown that compositions comprising crystalline calcium carbonate particles, in the form of coral exoskeleton, and a biocompatible polymer such as collagen, when supplemented with nerve growth agents, can effectively stimulate neural regeneration and repair in cultured rat hippocampal slices, resulting in migration of astrocytes and neuroblasts from the brain slices. In vivo, implantation of a composition comprising coral exoskeleton particles and collagen into the site of cortical injury in mice led to accumulation of neural cells and pre-synaptic structures within the wound (Example II). Supplementation of the compositions with nerve growth agents (Example II) stimulated massive cell growth within the cortical wounds and appearance of pre-synaptic and postsynaptic structures. Compositions comprising coral exoskeleton particles and collagen, supplemented with platelets and a platelet activator (Example II) effectively reduced the degree of glial scarring in the cortical lesion as well as stimulating neurogenesis within the implanted composition and surrounding tissue. Compositions comprising coral exoskeleton particles and collagen supplemented with platelets and insulin greatly enhanced recovery of functional deficits following traumatic brain injury (Example IV).

Thus, according to one aspect of the present invention there is provided a method for regenerating or repair of neural tissue comprising contacting the neural tissue with a composition comprising porous crystalline calcium carbonate or calcium phosphate particles and a biocompatible polymer.

The term “calcium carbonate” as used herein, refers to the chemical compound CaCO₃. Crystalline forms of calcium carbonate include, but are not limited to, aragonite, calcite, ikaite, vaterite and monohydrocalcite. The term “calcium phosphate” as used herein, refers to chemical salts of calcium and phosphate. Crystalline forms of calcium phosphate include, but are not limited to monocalcium phosphate Ca(H₂PO₄), dicalcium phosphate CaHPO₄, tricalcium phosphate Ca₃(PO₄)₂, hydroxyapatite Ca₅(PO₄)₃(OH), apatite Ca₁₀(PO₄)₆(OH, F, Cl, Br)₂, octacalcium phosphate and biphasic calcium phosphate. In some embodiments of the present invention the crystalline calcium phosphate comprises hydroxyapatite (hydroxylapatite), a main component of bone mineralization in mammals.

Calcium carbonate useful for the present invention can be obtained from natural sources, or prepared chemically. Natural sources of calcium carbonate include, but are not limited to rock formations, such as limestone, chalk, marble, travertine and tufa. Calcium carbonate is also a principle structural component of many life forms, and thus can be obtained from, inter alia, corals, plankton, coralline algae, sponges, brachiopods, echinoderms, bryozoa, mollusks and other calcium carbonate-containing organisms.

In some embodiments the calcium carbonate comprises aragonite.

As used herein, the term “aragonite” refers to the crystalline form of calcium carbonate, which can be commonly found in as mineral deposits in caves and in oceans, and in the shells of mollusks and exoskeleton of cold and warm-water corals.

In some embodiments, the calcium carbonate comprises calcite.

In some embodiments, the calcium carbonate comprises both aragonite and calcite.

In one embodiment, the aragonite comprises a coral exoskeleton. The term “coral exoskeleton”, as used herein, refers to the exoskeleton of marine madreporic corals. Natural coral (e.g. Porites) is composed of a mineral phase, principally calcium carbonate in the structural form of aragonite or calcite with impurities, such as Sr, Mg and F ions, and an organic matrix. Coral exoskeleton includes calcium carbonate in the form of aragonite or calcite, with or without additional components (minerals, organic and inorganic components) derived from or secreted by the living coral or life forms associated therewith.

Coral exoskeleton is also commercially available (e.g. Biocoral™) and has been reported to be biocompatible and resorbable. Coral-derived material described as coralline HA prepared by hydrothermally converting the original calcium carbonate of the coral Porites in the presence of ammonium phosphate, maintaining the original interconnected macroporosity of the coral, is also commercially-available (Pro Osteon®, Interpore Cross). The high content calcium carbonate coral exoskeleton has since been shown to be biocompatible and biodegradable at variable rates depending on porosity, the implantation site and the species.

In some embodiments of the invention, the coral exoskeleton or compositions comprising the same are derived from a coral. In one embodiment, the coral can comprise any species, including, but not limited to any one or more of the following species: Favites halicora; Goniastrea retiformis; Acanthastrea echinata; Acanthastrea hemprichi; Acanthastrea ishigakiensis; Acropora aspera; Acropora austera; Acropora sp. “brown digitate”; Acropora carduus; Acropora cerealis; Acropora chesterfieldensis; Acropora clathrata; Acropora cophodactyla; Acropora sp. “danai-like”; Acropora divaricata; Acropora donei; Acropora echinata; Acropora efflorescens; Acropora gemmifera; Acropora globiceps; Acropora granulosa; Acropora cf hemprichi; Acropora kosurini; Acropora cf loisettae; Acropora longicyathus; Acropora loripes; Acropora cf lutkeni; Acropora paniculata; Acropora proximalis; Acropora rudis; Acropora selago; Acropora solitaryensis; Acropora cf spicifera as per Veron; Acropora cf spicifera as per Wallace; Acropora tenuis; Acropora valenciennesi; Acropora vaughani; Acropora vermiculata; Astreopora gracilis; Astreopora myriophthalma; Astreopora randalli; Astreopora suggesta; Australomussa rowleyensis; Coscinaraea collumna; Coscinaraea crassa; Cynarina lacrymalis; Distichopora violacea; Echinophyllia echinata; Echinophyllia cf echinoporoides; Echinopora gemmacea; Echinopora hirsutissima; Euphyllia ancora; Euphyllia divisa; Euphyllia yaeyamensis; Favia rotundata; Favia truncatus; Favites acuticollis; Favities pentagona; Fungia granulosa; Fungia klunzingeri; Fungia mollucensis; Galaxea acrhelia; Goniastrea edwardsi; Goniastea minuta; Hydnophora pilosa; Leptoseris explanata; Leptoseris incrustans; Leptoseris mycetoseroides; Leptoseris scabra; Leptoseris yabei; Lithophyllon undulatum; Lobophyllia hemprichii; Merulina scabricula; Millepora dichotoma; Millepora exaesa; Millipora intricata; Millepora murrayensis; Millipore platyphylla; Monastrea curta; Monastrea colemani; Montipora caliculata; Montipora capitata; Montipora foveolata; Montipora meandrina; Montipora tuberculosa; Montipora cf vietnamensis; Oulophyllia laevis; Oxypora crassispinosa; Oxypora lacera; Pavona bipartita; Pavona venosa; Pectinia alcicornis; Pectinia paeonea; Platygyra acuta; Platygyra pini; Platygyra sp “green”; Platygyra verweyi; Podabacia cf lanakensis; Porites annae; Porites cylindrica; Porites evermanni; Porites monticulosa; Psammocora digitata; Psammocora explanulata; Psammocora haimeana; Psammocora superficialis; Sandalolitha dentata; Seriatopora caliendrum; Stylocoeniella armata; Stylocoeniella guentheri; Stylaster sp.; Tubipora musica; Turbinaria stellulata; or any coral known in the art, or a combination thereof.

In some embodiments, the coral is from the Porites, Acropora or Millepora species or a combination thereof. In one embodiment, the coral is from the Porites species. In one embodiment, the coral is Porites lutea. In another embodiment, the coral is from the Acropora species. In another embodiment, the coral is Acropora grandis (which in one embodiment is very common, fast growing, and easy to culture). Acropora samples can be easily collected in sheltered areas of the coral reefs and/or can conveniently be cultured.

In another embodiment, the coral is from the Millepora species. In one embodiment, the coral is Millepora dichotoma, which can be cloned and cultured, making Millepora useful in the compositions and methods of this invention.

In yet another embodiment, coral for use in compositions or methods of this invention include, but are not limited to Madreporaria, Helioporida of the order Coenothecalia, Tubipora of the order Stolonifera, Millepora of the order Milleporina, or others known in the art. In some embodiments, coral for use in the compositions and methods of this invention may comprise scleractinian coral, including in some embodiments, Goniopora and others. In some embodiments, coral for use in the compositions and methods of this invention may comprise Alveopora or bamboo corals, including in some embodiments, coral from the family Isididae, genera Keratosis, Isidella, and others.

Crystalline calcium carbonate or calcium phosphate suitable for use with the present invention comprises a porous form of crystalline calcium carbonate or calcium phosphate. In some naturally occurring porous crystalline calcium carbonate, for example, as found in coral exoskeletons, the pore size ranges from 1 micron to one millimeter. Thus, a specific example of average pore size (pore diameter) of porous crystalline calcium carbonate or calcium phosphate suitable for use in the compositions or methods of the invention is in the range of 1 micron-1 millimeter (also referred to herein as microporous). In one embodiment, the average pore size of the crystalline calcium carbonate or calcium phosphate is 30-180 microns. In another embodiment, the average pore size of the crystalline calcium carbonate or calcium phosphate is 15-500 microns. In another embodiment, the average pore size of the crystalline calcium carbonate or calcium phosphate is 150-220 microns. In one embodiment, the average pore size of the crystalline calcium carbonate or calcium phosphate is 250-1000 microns. In one particular embodiment the crystalline calcium carbonate or calcium phosphate is a microporous coral exoskeleton.

Aragonite suitable for use in compositions and/or methods of the invention may be prepared from coral or coral fragments, or from coral sand. Briefly, the coral can be prepared as follows: in one embodiment, coral or coral sand is purified from organic residues, washed, bleached, frozen, dried, sterilized or a combination thereof prior to use in the compositions and/or methods of the invention.

The crystalline calcium carbonate or calcium phosphate particles of the invention can be provided in a variety of forms, shapes and structures, compatible with many different applications of the invention. Some suitable forms and shapes can include, but are not limited to, for example, layered particles, blocks, spherical and hollow spherical particles, concentric spheres, rods, symmetrical and asymmetrical forms, amorphous and other irregular shaped particles. The crystalline calcium carbonate or calcium phosphate particles can be shaped, for example, to vary (increase or decrease) the surface area of the particles, or to allow predetermined minimal distances between two adjacent particles.

In some embodiments, preparation of the crystalline calcium carbonate or calcium phosphate (for example, aragonite or coral exoskeleton) includes contacting the crystalline calcium carbonate or calcium phosphate (e.g. aragonite, coral exoskeleton) of a desired size and shape, (for example, particulate) with a solution comprising an oxidizing agent, and washing and drying the crystalline calcium carbonate or calcium phosphate.

In some embodiments, the oxidizing agent for use in the processes of this invention may be any suitable oxidizing agent, for example, any oxidizing agent which facilitates the removal of organic debris from coral exoskeletons. In some embodiments, the oxidizing agent is sodium hypochlorite.

For most therapeutic applications, it is desirable that the calcite, or aragonite, when derived from natural sources, such as coral, be devoid of any cellular debris or other organisms associated therewith in its natural state. Thus, in some embodiments, the coral exoskeleton is an acellular coral exoskeleton.

According to this aspect, and in some embodiments, the process comprises conducting said contacting under mildly acidic conditions.

In one embodiment, calcium carbonate, aragonite or coral suitable for use in the compositions and/or methods of the invention is produced from coral or coral sand according to a process comprising washing ground solid calcium carbonate (e.g. aragonite), such as coral or naturally occurring coral sand with water to desalinate it, then disinfecting and drying the desalinated coral sand at temperatures of about 80 degrees to about 150 degrees C., preferably 90 degrees to 120 degrees C., cutting larger pieces of coral into small pieces, and grinding the disinfected and dried coral or coral sand into small particles, including but not limited to particles of 1-10 microns. In some embodiments, coral is ground into particles having a particle diameter in the range of 1-5, 1-20, 1-50, 1-100, 5-10, 10-15, 15-20, 10-50, 10-100, 20-100, 50-100, 80-150, 100-200, 100-350 or 150-500 microns across, and a particle volume in the range of 1-100, 50-500, 250-1000, 500-2500, 1000-5000 and 2500-10,000 cubic micron to 0.01-0.1, 0.05-0.5, 0.5-0.75, 0.75-1.0, 1.0-2.0 and 1.0-5.0 cubic millimeters in volume. In a particular embodiment, the particle diameter of the crystalline calcium carbonate particles is in the range of 1 micron to 5 millimeters. In specific embodiments, the particle diameter of the crystalline calcium carbonate particles is in the range of 85-225 microns across, 95-200 microns across, 100-200 microns across, 110-180 microns across, and 125-150 microns across. In some embodiments, the particle diameter of the crystalline calcium carbonate or calcium phosphate particles is 95, 105, 110, 120, 130, 140, 150, 160, 170, 180, 190, 200 or 210 microns across.

The present inventors have found that incorporation of coral skeleton particles in a biocompatible polymer (e.g. collagen gel) causes a dramatic elevation of the capacity of the coral skeleton particles to regenerate nervous tissue, for example, by enhancing accumulation of cells and neuronal structures such as neurites and synaptic structures (see Example III, hereinbelow). Thus, according to one aspect of the present invention, the neural tissue is contacted with a composition comprising porous crystalline calcium carbonate or calcium phosphate particles and a biocompatible polymer.

As used herein, the term “biocompatible polymer” refers to the ability of a polymer to be in contact with (be used with, near or integrated within) a living system, without adverse effect. Biocompatibility can be assessed by the presence or absence (or degree of severity) of any number of adverse effects, for example, but not limited to stimulation or exacerbation of host immune response, toxicity, disruption of tissue and/or organ function, metabolic changes, and the like. Additional criteria for biocompatibility, relating to the intended therapeutic function of the polymer include the ability of the polymer to perform its desired function with respect to a therapy, without eliciting any undesirable local or systemic effects in the recipient or beneficiary of that therapy, and the ability to generate the most appropriate beneficial cellular or tissue response in that specific situation, optimizing the clinically relevant performance of the polymer.

In one embodiment of this invention, the polymer is biodegradable. As used herein, the term “biodegradable” refers to a material which is degraded in the biological environment of the subject in which it is found. In some embodiments the biodegradable material undergoes degradation into its component subunits, via, for example, digestion, by a biochemical process. In other embodiments, biodegradation involves cleavage of bonds (whether covalent or otherwise), for example in a polymer backbone or side chain of the polymer.

In some embodiments, the porous crystalline calcium carbonate or calcium phosphate particles are biodegradable. In other embodiments, where it is advantageous for the composition to gradually be eliminated as the regeneration and/or repair of the neural tissue proceeds, or wherein there is need for replacing or changing implanted compositions, the entire composition of the invention is biodegradable, i.e. both the porous crystalline calcium carbonate or calcium phosphate particles and the polymer are biodegradable. Still further, in some embodiments, biodegradability of the composition can be engineered, providing an opportunity to match the projected life expectancy of the composition (e.g., an implant) in the tissue following implantation, to the requirements of the therapy. For example, the inventors have found that while compositions and implants comprising crystalline calcium carbonate particles on the small end of the size range (e.g. 5-20 microns in diameter) persisted for a shorter time when implanted in the wounds, as compared to compositions and implants having larger particle size, the life expectancy, after implantation, of compositions comprising such smaller particles is increased when the polymer (e.g. collagen) is added to the composition. Varying the density, porosity or other parameter of the polymer may also affect the degree of biodegradability (and thus the effective “life expectancy”) of the implant or composition when implanted in the wound. Without wishing to adhere to a specific hypothesis, it is conceivable that increased density, and lower porosity of the polymer component of the composition can reduce biodegradation of the calcium carbonate or calcium phosphate particles in the composition.

In one embodiment of this invention, the biocompatible polymer of the composition can be a permeable polymer, allowing passage of solutes throughout the volume of the composition. In one embodiment, the term “permeable” refers to having pores and openings which allow entry and/or exit of nutrients, therapeutic compounds, cell populations, or a combination thereof. As used herein, the terms “permeable” and “porous” are used interchangeably. Permeability of a biocompatible polymer can be a function of the presence of actual pores (openings, holes) within the polymer structure, and also, but not necessarily, permeability can be a function of the degree of cross linking of the linear structures within the polymer, for example, cross linking of collagen polypeptides to form collagen fibrils. In some embodiments, the amount of polymer molecules per volume, and/or degree of cross-linking of the polymer determine the “density” of the polymer, which in turn is typically in an inverse ratio to the permeability of the polymer. Permeability can be expressed, relative to the (solute) molecules which the polymer barrier either allows or excludes.

In some embodiments, the polymer can be prepared as a stock solution, diluted to desired density (e.g. w/v), and then mixed with the crystalline calcium carbonate or calcium phosphate particles to the desired polymer:particle ratio. For example, the polymer can be prepared at densities in the range of 0.5-15 mg/ml, 1-12 mg/ml, 2-10 mg/ml, 3-8 mg/ml, 2-6 mg/ml and 4-7 mg/ml, and specifically, in densities of 0.5, 1.0, 1.5, 2.0, 2.5, 3.0, 3.5, 4.0, 4.5, 5.0, 5.5, 6.0, 6.5 or 7.0 mg/ml polymer in a suitable solution (e.g. water, buffer (e.g. PBS), salt solutions, as desired/required for further use with the target neural tissue. These densities of polymer are particularly suitable for use when the polymer is collagen. In a specific embodiment, the biocompatible polymer is collagen, provided at a density of 4 mg/ml.

In some embodiments, the biocompatible polymer of the composition is uniform in density throughout the composition. For example, where density of the polymer is defined in terms of weight/volume (e.g. mg polymer/ml composition), the polymer component of the composition will be of the same weight polymer/volume composition throughout the composition. In other embodiments, the biocompatible polymer of the composition comprises polymers having a plurality of densities. Where the compositions comprise a plurality of densities, the composition can be designed so that the polymer of the outer surface of the composition (e.g. the surface in contact with the surrounding environment) is of higher density (e.g. greater degree of cross-linking), while the polymer of the inner portions of the composition is of a lower density (e.g. lesser degree of cross linking), or vice-versa (greater density polymer at inner portions, lower density polymer at outer surfaces). In other embodiments, the density of the polymer can vary from location to location throughout the composition. Variation in polymer density can be achieved in numerous ways, for example, by creating and combining distinct layers of differing polymer densities during the formation of the composition, by creating polymers having a gradient(s) of densities, gradual or sharply delineated, and the like. Addition of polymer having a greater or lesser density can take place onto, or even into predetermined space within the composition following initial formation of the composition, creating a composition with a polymer having a plurality of densities.

In some embodiments, the biocompatible polymer can be a film, or can be organized as particles.

In some embodiments, the biocompatible polymer of the composition of the invention comprises a natural polymer. Exemplary natural polymers include, but are not limited to collagen, albumin, elastin, silk, chitosan, agarose, alginate, fibrin, gelatin, cellulose, gluten, starch, sclerolutan, elsinan, pectin, galactan, curdlan, gellan, levan, emulsan, dextran, pullulan, heparin, chondroitin-6-sulfate, hyaluronic acid (HA) and combinations thereof.

In one embodiment, the polymer comprises synthetically modified natural polymers, and may include cellulose derivatives such as alkyl celluloses, hydroxyalkyl celluloses, cellulose ethers, cellulose esters, nitrocelluloses, and chitosan. Examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate and cellulose sulfate sodium salt. Further examples of natural and modified natural polymers suitable for use in the composition of the present invention are disclosed in detail in, for example, US Patent publication US 20030170474 to Qiao Teicheng et al.

In one embodiment, of this invention, a polymer comprises a synthetic biodegradable polymer. In one embodiment of this invention, a synthetic biodegradable polymer comprises alpha-hydroxy acids including poly-lactic acid, polyglycolic acid, enantioners thereof, copolymers thereof, polyorthoesters, and combinations thereof. In some embodiments, the biodegradable polymers are co-polymers of natural polymers and synthetic polymers.

In one embodiment, a polymer of this invention comprises a poly(cianoacrylate), poly(alkyl-cianoacrylate), poly(ketal), poly(caprolactone), poly(acetal), poly(.alpha.-hydroxy-ester), poly(.alpha.-hydroxy-ester), poly(hydroxyl-alkanoate), poly(propylene-fumarate), poly(imino-carbonate), poly(ester), poly(ethers), poly(carbonates), poly(amide), poly(siloxane), poly(silane), poly(sulfide), poly(imides), poly(urea), poly(amide-enamine), poly(organic acid), poly(electrolytes), poly(p-dioxanone), poly(olefin), poloxamer, inorganic or organometallic polymers, elastomer, or any of their derivatives, or a copolymer combination thereof.

In one embodiment, a polymer of the invention comprises poly(D,L-lactide-co-glycolide) (PLGA). In another embodiment, the polymer comprises poly(D,L-lactide) (PLA). In another embodiment, the polymer comprises poly(D,L-glycolide) (PGA). In one embodiment, the polymer comprises a glycosaminoglycan. In one embodiment, the polymer comprises synthetic degradable polymers, which may include, but are not limited to polyhydroxy acids, such as poly(lactide)s, poly(glycolide)s and copolymers thereof; poly(ethylene terephthalate); poly(hydroxybutyric acid); poly(hydroxyvaleric acid); poly[lactide-co-(.epsilon.-caprolactone)]; poly[glycolide-co(.epsilon.-caprolactone)]; poly(carbonate)s, poly(pseudo amino acids); poly(amino acids); poly(hydroxyalkanoate)s; poly(anhydrides); poly(ortho ester)s; and blends and copolymers thereof.

In one embodiment of this invention, a polymer comprises proteins such as zein, modified zein, casein, gelatin, gluten, serum albumin, collagen, actin, alpha-fetoprotein, globulin, macroglobulin, cohesin, laminin, fibronectin, fibrinogen, osteocalcin, osteopontin, osteoprotegerin, or others, as will be appreciated by one skilled in the art. In another embodiment, a polymer may comprise cyclic sugars, cyclodextrins, synthetic derivatives of cyclodextrins, glycolipids, glycosaminoglycans, oligosaccharide, polysaccharides such as alginate, carrageenan (chi, lamda, mu, kappa), chitosan, celluloses, condroitin sulfate, curdlan, dextrans, elsinan, furcellaran, galactomannan, gellan, glycogen, arabic gum, hemicellulose, inulin, karaya gum, levan, pectin, pollulan, pullulane, prophyran, scleroglucan, starch, tragacanth gum, welan, xanthan, xylan, xyloglucan, hyaluronic acid, chitin, or a poly(3-hydroxyalkanoate)s, such as poly(beta-hydroxybutyrate), poly(3-hydroxyoctanoate) or poly(3-hydroxyfatty acids), or any combination thereof.

In some embodiments, the polymer is a collagen. It will be noted that many different types of collagen are known in the art- “Fibrillar” collagen (Type I, II, III, V, XI), “Facit” collagen (Type IX, XII, XIV), Short chain collagen (Type VIII, X), “Basement membrane” (Type IV), “Other” (Type VI, VII, XIII) In a particular embodiment, the polymer comprises type I collagen.

In one embodiment, a polymer may comprise chemical derivatives thereof (substitutions, additions, and elimination of chemical groups, for example, alkyl, alkylene, hydroxylations, oxidations, and other modifications routinely made by those skilled in the art), blends of, e.g. proteins or carbohydrates alone or in combination with synthetic polymers.

In one embodiment, the crystalline calcium carbonate particles of the invention are covalently associated with the polymer via the use of a cross-linking agent. In one embodiment, the phrase “cross-linking agent” refers to an agent which facilitates the formation of a covalent bond between 2 atoms. In some embodiments, the cross-linking agent is a zero-length cross-linking agent.

Cross-linking agents suitable for use with the compositions of the present invention are well known in the art. In one embodiment, the cross-linking agent is (1 ethyl 3-(3-dimethyl aminopropyl)carbodiimide (EDAC), N-Sulfohydroxy succinamide (Sulfo NHS), 5-iodopyrimidines, N-carbalkoxydihydroquinolines, pyrroloquinolinequinones, or combinations thereof.

In one embodiment, the cross-linking agent is a homobifunctional cross-linker, such as, for example, a N-hydroxysuccinimide ester (e.g. disuccinimidyl suberate or dithiobis(succinimidylpropionate), homobifunctional imidoester (e.g. dimethyladipimidate or dimethyl pimelimidate), sulfhydryl-reactive crosslinker (e.g. 1,4-di-[3′-(2′-pyridyldithio)propionamido]butane), difluorobenzene derivative (e.g. 1,5-difluoro-2,4-dinitrobenzene), aldehyde (e.g. formaldehyde, glutaraldehyde), bis-epoxide (e.g. 1,4-butanediol diglycidyl ether), hydrazide (e.g. adipic acid dihydrazide), bis-diazonium derivative (e.g. o-tolidine), bis-alkylhalide, or a combination thereof.

In one embodiment, the cross-linking agent is a heterobifunctional cross-linker, such as, for example, an amine-reactive and sulfhydryl-reactive crosslinker (e.g. N-succinimidyl 3-(2-pyridyldithio)propionate, a carbonyl-reactive and sulfhydryl-reactive crosslinker (e.g. 4-(4-N-maleimidophenyl)butyric acid hydrazide), or a combination thereof.

In some embodiments, the cross-linking agent is a trifunctional cross-linkers, such as, for example, 4-azido-2-nitrophenylbiocytin-4-nitrophenyl ester, sulfosuccinimidyl-2-[6-biotinamido]-2-(p-azidobenzamido)hexanoamido]ethyl- -1,3′-dithiopropionate (sulfo-SBED), or a combination thereof.

In another embodiment, the cross-linking agent is an enzyme. In one embodiment of this invention, the cross-linking agent comprises a transglutaminase, a peroxidase, a xanthine oxidase, a polymerase, or a ligase, or a combination thereof. The choice of concentration of the cross-linking agent utilized for activity will vary, as a function of the volume, agent and polymer chosen, in a given application, as will be appreciated by one skilled in the art. In some embodiments, collagen can be crosslinked by combining collagen with chondroitin sulfate to form a slurry, which can then be crosslinked dehydrothermally (heat under vacuum), by glutaraldehyde, ethanol and/or by UV treatment.

The degree of cross-linking can be assessed chemically, biochemically and physically—for example, by measuring the shrinkage temperature (Tₛ) of the polymer, for example, of cross-linked collagen.

In one embodiment, the association of the crystalline calcium carbonate or calcium phosphate particles of this invention with a polymer of this invention comprises a physical and/or mechanical association. For example, in one embodiment, a physical and/or mechanical association may comprise imbibing of any means, air drying, using a cross-linking agent, applying of heat, applying vacuum, applying lyophilizing methods, applying freezing, applying centrifuge, applying mechanical forces or any combination thereof, to promote the physical and/or mechanical association between the crystalline calcium carbonate or calcium phosphate particles and a polymer as described herein.

It will be apparent to one skilled in the art that the physical and/or mechanical and/or chemical properties of a polymer and components thereof may influence methods of use of this invention and kits thereof, for inducing or enhancing neural tissue repair and regeneration.

In one embodiment, the composition of this invention has a thickness of between 0.01 μm and 2.0 μm. In one embodiment, the composition has a thickness of about 1.0 μm. In one embodiment, the composition of this invention has a thickness of between 10 μm and 50 um. In one embodiment, the composition has a thickness of about 10-25, or about 15-30, or about 25-50 μm. In one embodiment, the composition has a thickness of about 50-80, or about 60-90, or about 80-120 μm. In one embodiment, the composition has a thickness of about 100-150, or about 130-200, or about 150-250 μm. In one embodiment, the composition has a thickness of about 200-350, or about 300-600, or about 450-1000 μm. Larger compositions, having thickness of about 1000-5000 microns are also envisioned. In some embodiments, multiple compositions (a plurality of compositions) comprising polymers are implanted into a repair site, wherein the thickness of a first composition may vary as compared to a polymer thickness of a second composition (or third composition or more) implanted in the repair site.

In one embodiment, the thickness of the composition influences physical characteristics of a composition of this invention. For example, the thickness of the composition may influence elasticity, tensile strength, adhesiveness, or retentiveness, or any combination thereof of a composition of this invention. In one embodiment, the thickness of the composition increases the elasticity of a composition of this invention. In one embodiment, thickness of the composition increases the tensile strength of a composition of this invention.

It will be appreciated that affinity of the composition of the invention, for certain types of cells, is a significant factor in the regeneration and/or repair of the neural tissue. While not wishing to be limited to a single hypothesis, one explanation could be that the extent of cell-substrate suitable surface area, and the cell substrate character thereof, are correlated with the degree of cell migration and neural outgrowth from the tissue in the vicinity of the wound. In one embodiment, the affinity of the composition relates to affinity of neural cells or other stem cells, blood cells, blood vessels, tissue at a site of neural tissue repair or neural tissue. In one embodiment, a polymer may decrease the affinity of the composition of this invention for cells, and in another embodiment, a polymer may increase the affinity of a composition of the invention for cells. One skilled in the art will recognize that a polymer may increase affinity for an item (e.g. type of neural cell) while decreasing affinity for another item (e.g. scar tissue). For example, in some embodiments, the cell population having affinity with the composition and retained within the composition of the invention is an astrocyte population. In another embodiment, the cell population having affinity with the composition and retained within the composition is a neuroblast population. In yet another embodiment, the retentiveness of the composition relates to retention of effector compounds, such as neural growth agents.

The present inventors have found that contact of the neural tissue cells within a recovering wound with implanted coral exoskeleton particles enhances the survival rate and degree of neural cell differentiation (expression of astrocytic and neuronal markers) of the wounded neural tissue. Thus, the distribution of crystalline calcium carbonate or calcium phosphate particles within the compositions of the invention can affect the function of the composition in neural tissue regeneration and repair. Thus, in some embodiments, the crystalline calcium carbonate or calcium phosphate particles are distributed evenly within the biocompatible polymer, i.e. with relatively uniform distances between adjacent calcium carbonate or calcium phosphate particles, in three dimensions, within the composition. In other embodiments, the crystalline calcium carbonate or calcium phosphate particles are distributed unevenly within the biocompatible polymer, i.e. with clustering or concentrations of calcium carbonate particles and varying distances between adjacent particles within the composition. Where the calcium carbonate or calcium phosphate particles are organized in clusters, the clusters in turn may be evenly distributed or unevenly distributed throughout the composition of the invention. Inter-particle distances within the compositions of the invention can be in the range of 0.5 μm-45 μm, including but not limited to 1-40 μm, 2-35 μm, 5-30 μm, 7.5-20 μm, and 10-15 μm. Where the calcium carbonate particles are non-evenly distributed, but organized in clusters, suitable inter-cluster distances include distances in the range of 200-1500 μm, 250-1250 μm, 400-1000 μm, 500-850 μm, 600-650 μm, 1000-1500 μm.

The compositions of the present invention can be produced so that there is a concentration of crystalline calcium carbonate particles on a surface of the composition, affording exposure of the surrounding medium and cells of, for example, damaged neural tissue within a wound, to a higher density of particles. Conversely, it may be advantageous for some of the compositions of the present invention to retain a greater portion of the calcium carbonate particles within the polymer of the composition, in order to minimize the biodegradation of the particles and/or to increase the exposure of the cells to the surrounding gel.

Thus, in some embodiments, the composition comprises the crystalline calcium carbonate particles distributed on an external surface thereof. It will be appreciated that in compositions of the invention which are fashioned in solid geometric shapes (spheres, cylinders, rods, etc), the external surface is the outer perimeter. Wherein the compositions are fashioned into more complex configurations (e.g. toroid, lattice and the like), both internally located and external surfaces can be exposed to the surrounding medium, and these surfaces can incorporate the crystalline calcium carbonate particles wherever predetermined or desired.

The method of the present invention can be used to regenerate or repair neural tissue. Contacting neural tissue of experimentally created cortical lesions with the compositions of the invention comprising crystalline calcium carbonate particles, in the form of coral exoskeleton, and a biocompatible polymer, in the form of collagen, promoted neuronal migration from surrounding neural tissue and synapse formation within the wounded tissue (see Example II). Thus, in some embodiments, the present invention can be used to regenerate and/or repair neural tissue.

Neural tissue is the main component of the nervous system, including the brain and spinal cord of the central nervous system and the branching peripheral nerves of the peripheral nervous system. Neural tissue is a highly differentiated tissue comprised of neurons, which are made up of nerve cell bodies, with their projecting axons and dendrites, and neuroglia, which is a supporting medium for the neurons. In the central nervous system, the neuroglia is made up of microglia cells, astrocytes, oligodendrocytes and NG2 glia cells. Glia of the peripheral nervous system comprises Schwann cells, providing a myelin sheath for the axons of the neurons, and satellite cells. Contacting neural tissue with the composition of the invention can be effective in regenerating and/or repairing all of the cell types comprising the neural tissue. Thus, in some embodiments, the method of the invention can be used to regenerate or repair neural tissue selected from the group consisting of neuronal tissue, glial tissue, central nervous system neural tissue and peripheral nervous system neural tissue.

The inventors have shown that the methods and compositions of the invention are particularly effective for repair and regeneration of central nervous system lesions (brain injury). Thus, in a particular embodiment, the neural tissue is central nervous system tissue, and the method comprises contacting the central nervous system tissue with the compositions of the invention. In a specific embodiment, the neural tissue is brain tissue. When the contacting is contacting with brain tissue, contacting can be with any portion of the brain tissue, including, but not limited to the cerebrum (e.g. cerebral cortex), cerebellum, and brainstem (e.g. thalamus, medulla, midbrain). The methods and compositions of the invention are also suitable for administration to other portions of the central nervous system (e.g. spinal cord) and peripheral nervous system (e.g. nerves and ganglia of the somatic and autonomic nervous systems). In some embodiments, the nervous tissue is selected from the group consisting of: nervous tissue of an embryonic organism; nervous tissue of a fetal organism, nervous tissue of a newborn (in humans, up to 28 days old), nervous tissue of an infant (in humans from 29 days to 1 year old; nervous tissue of a young organism (in humans, about 1 to about 9 years); nervous tissue of an adolescent organism (in humans about 9 to about 14 years); nervous tissue of a young adult organism (in humans, about 15 to about 30 years); nervous tissue of an adult organism (in humans about 30 to about 70 years); and nervous tissue of an aged organism (in humans, about 70 years and above).

Application of the compositions comprising crystalline calcium carbonate particles and biocompatible polymer in the brain can be performed by direct administration, e.g. topically applying the compositions of the invention to the neural tissue, or by intravenous/intra-arterial injection of the compositions at or near an area of brain tissue in need of regeneration and/or repair, i.e. damaged, injured and/or diseased brain neural tissue. Other means of delivering the compositions of the invention intravascularly to the desired region of brain neural tissue can also be employed (e.g. cannulae). Similarly, the compositions of the invention can be contacted with other central nervous system neural tissue (e.g. spinal cord) and peripheral neural tissue by direct topical application, intravenous/intra-arterial injection or intravascularly, as described.

In some embodiments, the composition of the invention can be provided in a gel, or semisolid consistency, which is particularly suited for application to damaged, injured or diseased neural tissue following a local loss or disruption of neural tissue, resulting in an artificial space within the neural tissue. The composition of the invention, when a gel or semisolid, can be applied directly to such a space, and formed to substantially fill the space with the composition. Both the polymer and particular components of the composition contribute to providing mechanical support for prevention of structural collapse of the wound or lesion during recovery and repair.

In some embodiments, regenerating or repairing of neural tissue comprises contacting the neural tissue with the composition of the invention, and further contacting the neural tissue with a nerve growth or regeneration agent. Contacting the neural tissue with the nerve growth or regeneration agent can be performed prior to, concomitant with, or following the contacting with the composition of the invention.

In some embodiments, the contacting with the nerve growth or regeneration agent is concomitant with contacting with the composition of the invention. Yet further, the composition of the invention may comprise the nerve growth or regeneration agent.

As used herein, the phrase “nerve growth or regeneration agent” relates to an agent which promotes growth, survival, regeneration (e.g. neurogenesis) and/or repair of neural tissue (e.g. neurons, glial tissue).

Nerve growth or regeneration agents include, but are not limited to neuroprotective or neurotrophic agents, growth factors, metabolic effectors and the like. For neurodegenerative disorders, such as, for example, Alzheimer's disease, Parkinson's disease and Huntington's disease, or other disease involving loss of locomotion or cognitive function such as memory, neuroprotective or neurotrophic agents are particularly suitable. The nerve growth or regeneration agent may be one that promotes neuronal survival, stimulates neurogenesis and/or synaptogenesis, rescues hippocampal neurons from beta-amyloid-induced neurotoxicity and/or reduces tau phosphorylation. Non-limiting examples of agents suitable for treating such neurodegenerative disorders, and neurological disorders, include luteinizing hormone releasing (LHRH) and agonists of LHRH, such as deslorelin; neurotrophic factors, such as those from the neurotrophin family, including nerve growth factor (NGF), brain-derived neurotrophic factor (BDNF), neurotrophin-3 and neurotrophin-4/5; the fibroblast growth factor family (FGFs), including acidic fibroblast growth factor and basic fibroblast growth factor; the neurokine family, including ciliary neurotrophic factor, leukemia inhibitory factor, and cardiotrophin-1; the transforming growth factor-p family, including transforming growth factor beta1-3 (TGF-betas), bone morphogenetic proteins (BMPs), growth/differentiation factors such as growth differentiation factors 5 to 15, glial cell line-derived neurotrophic factor (GDNF), neurturin, artemin, activins and persephin; the epidermal growth factor family, including epidermal growth factor, transforming growth factor-alpha and neuregulins; insulin and the insulin-like growth factor family, including insulin-like growth factor-1 (IGF-1) and insulin-like growth factor-2 (IGF-2); the pituitary adenylate cyclase-activating polypeptide (PACAP)/glucagons superfamily, including PACAP-27, PACAP-38, glucagons, glucagons-like peptides such as GLP-1 and GLP-2, growth hormone releasing factor, vasoactive intestinal peptide (VIP), peptide histidine methionine, secreting and glucose-dependent insulinotropic polypeptide; and other neurotrophic factors, including activity-dependent neurotrophic factor and platelet-derived growth factors (PDGFs). Such nerve growth or regeneration agents are also suitable for treating acute CNS (e.g. brain) injury and chronic CNS (e.g. brain injury) (neurogenesis). In a specific embodiment, the nerve growth or regeneration agent is selected from the group consisting of insulin, epidermal growth factor (EGF), brain derived neurotrophic factor (BDNF), glial cell-derived neurotrophic factor (GDNF), nerve growth factor (NGF), VGF nerve growth factor (VGF), neurotrophin 3 (NT3), and cytokines. In one embodiment, the nerve growth or regeneration agent is insulin. Insulin can be provided in any formulation or composition suitable for application to the neural tissue, or suitable for inclusion in the composition of the invention, for example, as, natural and synthetic insulin, zinc-insulin solution, insulin-protamine solution, insulin analogues and the like. Suitable concentrations of insulin for use in the compositions and methods of the present invention include, but are not limited to 1-50 μg/ml, 2-45 μg/ml, 4-40 μg/ml, 5-35 μg/ml, 8-30 μg/ml, 10-25 μg/ml, 12-20 μg/ml, 8, 9, 10, 11, 12, 13, 14, 15, 16, 17, 18, 19 and 20 μg/ml. In one particular embodiment, the insulin concentration is 12 μg/ml.

In one embodiment, the nerve growth or regeneration agent is epidermal growth factor (EGF). EGF can be provided in any formulation or composition suitable for application to the neural tissue, or suitable for inclusion in the composition of the invention, for example, as synthetic, natural or recombinant EGF in solution, or in a solid or semi-solid formulation. Suitable concentrations of EGF for use in the compositions and methods of the present invention include, but are not limited to 10-200 ng/ml, 20-180 ng/ml, 30-150 ng/ml, 40-135 ng/ml, 50-120 ng/ml, 60-100 μg/ml, 65-90 ng/ml, 55, 58, 60, 63, 66, 68, 70, 73, 75, 78, 80 and 85 ng/ml. In one particular embodiment, the EGF concentration is 60 ng/ml.

In a particular embodiment, the nerve growth and regeneration agents comprise insulin (12 μg/ml) and EGF (60 ng/ml). In other embodiments, the composition of the invention comprises added nerve growth and one or more regeneration agents selected from insulin and EGF. In further embodiments, the composition of the invention comprises both of insulin and EGF.

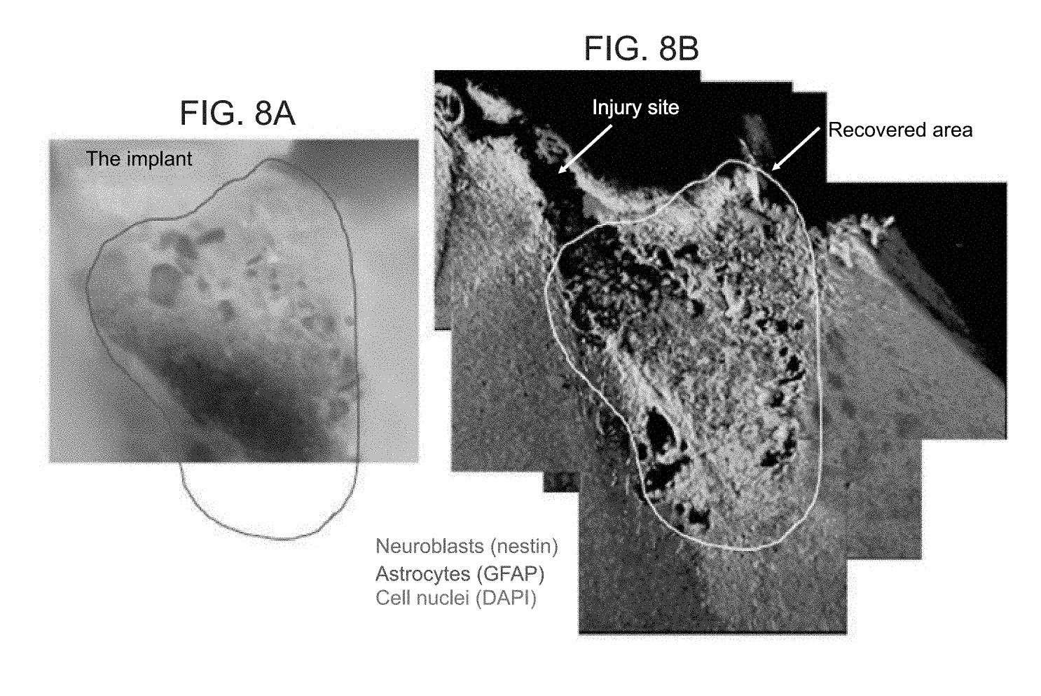

The present inventors have surprisingly shown that implantation of compositions comprising crystalline calcium carbonate, collagen and platelets (thrombocytes) into cortical wounds was effective in preventing astrocyte scar formation and enhancing neuroblast accumulation at the wound site (Example II, FIGS. 8A-B, and below). Thus, in some embodiments, the composition of the invention comprises added platelets (thrombocytes). Platelets suitable for use with the present invention can be obtained either by isolation from units of whole blood, or collected by platelet apheresis. Pooled whole blood platelets can be prepared in a number of ways, for example, by a “soft spin” of the blood unit in a centrifuge to separate red cells and platelet rich plasma, and then a faster spin to harvest the platelets, or by centrifuging the blood unit to produce a “buffy coat”, from which platelets can be isolated by further centrifugation. Apheresis platelets are collected using a mechanical device that draws blood from the donor and centrifuges the collected blood to separate out the platelets and other components to be collected. The remaining blood is returned to the donor. There are many advantages to this method, including the reduction in contaminating red blood cells and elimination of the need for multiple donors. In some embodiments, platelets can be incorporated into the composition of the invention. Suitable concentrations of platelets for use in the compositions and methods of the present invention include, but are not limited to a range [measured as platelets per 15 μl volume of the composition for implantation (for example, comprising collagen and coral skeleton particles)] of 1×10⁴-1×10⁶ platelets, 2×10⁴-8×10⁵ platelets, 5×10⁴-2×10⁵ platelets, 1×10⁵-5×10⁵ platelets and 1×10⁵-2×10⁵ platelets per 15 μl volume of composition. In one particular embodiment, the platelets are added to 1×10⁵-2×10⁵ platelets per 15 μl volume of the composition for implantation (for example, comprising collagen and coral skeleton particles). In some embodiments, a platelet-rich plasma is added to the compositions to provide platelets. In some embodiments, the implants comprise platelet-rich plasma providing 1×10⁵-5×10⁵ platelets per 15 μl volume of the composition for implantation. As used herein, the term “platelet-rich plasma” or “PRP” refers to a blood composition which has been enriched for the platelet fraction by centrifugation (at least once) and resuspended in plasma, as described above.

In some embodiments, addition of platelets to the composition of the invention further includes addition of adenosine di-phosphate (ADP), in order to stimulate activation of platelets. ADP can be added within the range of 10-40 mM. Specifically, ire some embodiments, the concentration of added ADP within the composition of the invention is 25 mM.

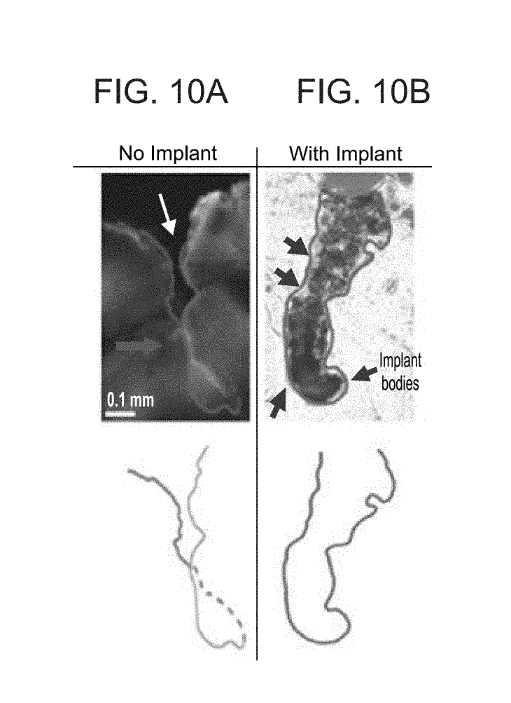

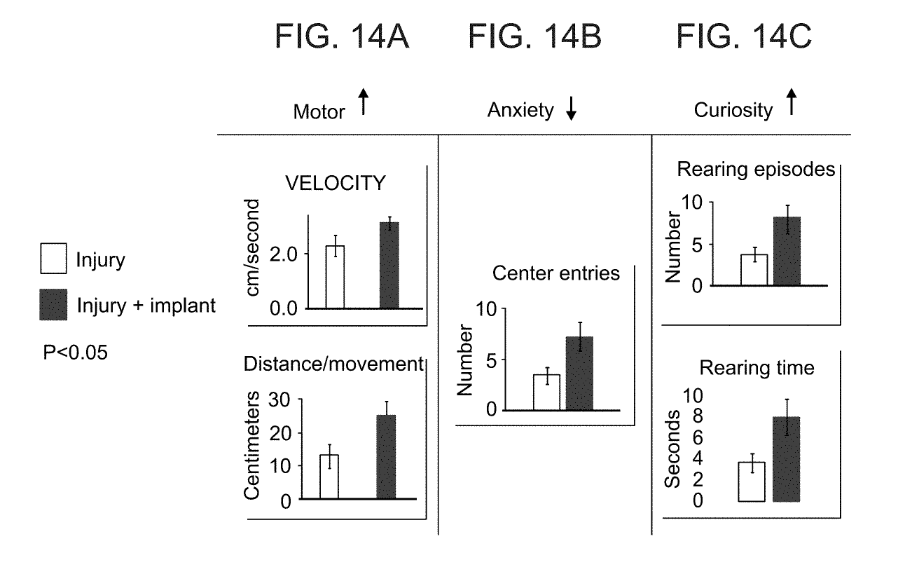

The present inventors have surprisingly shown that implantation of compositions comprising crystalline calcium carbonate, collagen, platelets (e.g. as platelet rich plasma) and insulin into cortical wounds was effective in enhancing neuroblast accumulation at the wound site (Example II, FIGS. 10A-B, 11A-B, 12A-B and 13B-C, below) and in recovery from function deficit following blunt cortical trauma (Example IV, FIG. 14A-C). Thus, in some embodiments, the composition of the invention comprises added platelets (thrombocytes) and insulin.

The compositions and methods of the invention can be used for treatment of neural tissue damaged by stroke. In the case of stroke treatment, the nerve growth or regeneration agent can be, for example, one that protects cortical neurons from nitric oxide-mediated neurotoxicity, promotes neuronal survival, stimulates neurogenesis and/or synaptogenesis and/or rescues neurons from glucose deprivation. Examples of such nerve growth or regeneration agents include, but are not limited to the neurotrophic factors previously described herein, active fragments thereof, as well as analogs and active fragments of the factors. Peptide growth factor mimetics of, and antagonists to, for example, EPO, granulocyte colony-stimulating factor (GCSF), and thrombopoietin useful in the invention can be screened for as reviewed by K. Kaushansky, Ann. NY Acad. Sci., 938:131-138 (2001) and as described for EPO mimetic peptide ligands by Wrighton et al., Science, 273(5274):458-450 (1996). The mimetics, agonists and antagonists to the peptide growth factors, or other peptides or proteins described herein, may be shorter in length than the peptide growth factor or other polypeptide that the mimetic, agonist or antagonist is based on.

Hormones that are suitable, in some instances, as nerve growth or regeneration agents in the context of the present invention include, but are not limited to melanocortin receptor (MCR) agonists and antagonists, hormone peptide YY (PYY), leptin and ghrelin, ciliary neurotrophic factor or analogs thereof, glucagon-like peptide-1 (GLP-1), insulin mimetics and/or sensitizers and dopaminergic, noradrenergic and serotinergic agents.

The nerve growth or regeneration agent can be neural-acting polypeptides such as growth hormone releasing factor, vasopres sin, and derivatives thereof, or therapeutic protein for treatment of autoimmune disorders, such as multiple sclerosis, includes interferons, including beta-interferon, and transforming growth factor betas.

Peptide nerve growth or regeneration agents may be human polypeptides, although the polypeptides may be from other species or may be synthetically or recombinantly produced. The original amino acid sequence may also be modified or reengineered such as for improved potency or improved specificity (e.g. eliminate binding to multiple receptors) and stability.

Polypeptide nerve growth or regeneration agents utilized herein may also be mimetics, such as molecules that bind to the same receptor but have amino acid sequences that are non-homologous to endogenous human peptides. For example, the peptide nerve growth or regeneration agents may include natural amino acids, such as the L-amino acids or non-natural amino acids, such as D-amino acids, the amino acids in the polypeptide may be linked by peptide bonds or, in modified peptides, including peptidomimetics, by non-peptide bonds.

Additional agents such as other growth factors, anti-inflammatory agents and/or antibiotics, which promote viability, proliferation, differentiation and/or migration of neural tissue cells, analgesics and the like can also be used with the compositions and methods of the invention. Some examples of compositions suitable for use as nerve growth and regeneration agents in the present invention are the compositions described in PCT Publication No. WO/2001/052878 to Eisenbach-Schwartz, et al., WO/2006/127712 to Palmore, et al., and Patent Application US2010/0303934, to Soumayanth et al.

Crystalline calcium carbonate, with or without biocompatible polymers can be used for culture of cells, including neural tissue cells. In some circumstances, addition of cultured or fresh neural tissue cells (e.g. neurons, glial cells, neural stem cells, etc) or stem cells to the damaged or injured neural tissue can be advantageous. Thus, in some embodiments, the composition of the invention can further comprise neural tissue cells, either freshly seeded neural tissue cells, or cells from neural tissue cell culture, or both. It will be appreciated that native, unmanipulated neural tissue cells, as well as genetically modified (transformed) neural tissue cells are suitable for use in the compositions and methods of the invention.

The method and composition of the invention can be used for regeneration and repair of neural tissue in a subject in need thereof. As used herein, the term “subject in need thereof” includes mammals, and specifically human beings at any age which suffer from a neurological or neurologically-associated pathology. This term also encompasses individuals who are at risk to develop such a pathology.

Thus, in some embodiments, the contacting of the composition with neural tissue is performed in vivo, in a subject in need thereof.

The subject in need thereof can be suffering from injured or damaged neural tissue. As used herein, the terms “injured” and “damaged” refer not only to a disrupted physical state of the neural tissue, but also to a disrupted functional state of the neural tissue, which may appear anatomically sound but suffer from absent or improper (excess or limited) neural transmission and/or signaling. In some embodiments, the neural injury or damage can be caused by a condition such as peripheral nerve injury or neuropathy (traumatic nerve injury, lower motor neuron lesion, demyelinating disease, diabetic neuropathy, and the like), cranial or cerebral trauma, aneurysm, spinal cord injury, stroke and disease. In a specific embodiment, the subject is suffering from traumatic brain injury (TBI). In some embodiments, the traumatic brain injury is a blunt trauma to the brain. In other embodiments, the traumatic brain injury is a cortical injury or cortical wound.

Some non-limiting examples of neural tissue disease suitable for treatment with the methods or compositions of the invention include Alzheimer's disease, Parkinson's disease, Huntington's disease, multiple sclerosis (MS), Creutzfeldt-Jacob's disease, kuru, multiple system atrophy, amyotropic lateral sclerosis (ALS, Lou Gherig's disease), progressive supranuclear palsy, optic neuritis, diabetic retinopathy, macular degeneration and glaucoma.

The composition of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound or combination of compounds to an organism.

Herein the term “active ingredient” refers to the compounds or combinations thereof accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Conventional approaches for delivery of therapeutic compositions to the central nervous system (CNS) include: neurosurgical strategies (e.g., intracerebral injection or intracerebroventricular delivery), the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a neural tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

Pharmaceutical compositions include aqueous compositions of the active preparation. Additionally, suspensions of the active ingredients may be prepared as appropriate oily or water based suspensions. Suitable lipophilic solvents or vehicles include fatty oils such as sesame oil, or synthetic fatty acids esters such as ethyl oleate, triglycerides or liposomes. Aqueous suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredients may be in powder form for constitution with a suitable vehicle, e.g., sterile, pyrogen-free water based solution, before use.

Pharmaceutical compositions suitable for use in context of some embodiments of the invention include compositions wherein the active ingredients are contained in an amount effective to achieve the intended purpose. More specifically, a therapeutically effective amount means an amount of active ingredients (crystalline calcium carbonate and biocompatible polymer) effective to prevent, alleviate or ameliorate symptoms of a disorder or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired migration of neurons out of cortical slices of rodent brain in culture. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide appropriate (neural tissue) levels of the active ingredient sufficient to induce or suppress the biological effect (minimal effective concentration, MEC). The MEC will vary for each preparation, but can be estimated from in vitro data. Dosages and treatment regimen necessary to achieve the MEC will depend on individual characteristics and route of administration. Detection assays can be used to determine plasma concentrations.

As noted above, the methods and compositions of the present invention can be particularly effective in preventing collapse of neural tissue wounds, a common problem in TBI repair, because the compositions, in a semi-solid or gel formulation can form to the contours of the wound and provide mechanical support. In some embodiments the composition of the invention can be prepared in vials, tubes or boxes in the form of ointment or foam, having a gel- or gel-like viscosity and moisture, so as to enable topical application along the entire area of the wound's walls and filling wounds of any size and shape. It can be apply using a simple mechanical applicator tool (e.g. spatula), pressure or spray.

It will further be appreciated that in many cases of neural tissue injury and/or disease, it is desirable to apply the compositions of the present invention before substantial formation of astrocytic scar tissue has taken place, e.g. as soon after the neural tissue has been damaged. In some embodiments, the compositions of the invention is applied immediately following the injury, while the wound is bleeding. Under such condition, the composition can be well exposed to brain cells capable of regenerating nervous tissue. It is conceivable that bleeding is advantageous for the recovery due to the regenerating role of various blood components such as plasma factors, platelets and leukocytes. Likewise, when treating a wound (e.g. TBI) with the compositions and methods of the present invention, a few hours to few days following wounding it is desirable to remove blood clots that can block scaffold-cells contact. When treating the wound seven or more days following the injury it may be necessary to surgically injure or remove the scar tissue, again, in order to expose the regenerating cells to the compositions of the invention.

Application of compositions and methods of the invention to a neural tissue would (e.g. brain injury) can result in regeneration of nervous tissue within few weeks from application, at a depth of few millimeter from the wound's walls. In some instances, a single thick piece of the composition can be sufficient to enable full wound recovery in a period of months to years. In other embodiments, recovery can proceed more slowly due to environmental factors (e.g. neurotrophic factors, oxygen supply). Thus, in some cases, some wounds (e.g. larger wounds, of the scale of centimeters) may require sequential (repeated) application of a plurality of thinner (e.g. millimeter thick) layers of the composition of the invention, and optionally additional factors as well.

Thus, in one embodiment, the composition is contacted with the neural tissue within 1 hour, 5 hours, 10 hours, 12 hours, 18 hours, 24 hours, 2 days, 3 days, 5 days, 7 days, 10 days, 12 days or 14 days following onset of the neural tissue damage. In a particular embodiment, the composition is contacted with the neural tissue no more than 14 days following onset of the neural tissue damage.

In some embodiments, particularly when the compositions of the present invention are applied to freshly wounded neural tissue, formation of blood clot or clots may be encountered. Since blood clots can provide a mechanical obstacle to neural tissue repair and regeneration, may prevent influx of factors important for neural tissue repair and regeneration and can be conducive to production of astrocytic scars, in some embodiments the composition of the present invention is contacted with the diseased or damaged neural tissue following removal of blood clots from the neural tissue. In older wounds (longer time from onset of neural tissue damage—e.g. >2 weeks), scar tissue will commonly have begun to form within the wound. In such cases, scar tissue formation can be disrupted (e.g. mechanically, chemically) before application of the composition, in order to allow for effect of the composition on the surrounding healthy neural tissue and migration of neural tissue into the wound. It will also be appreciated that repeated applications of the composition of the present invention may be required, depending on outcome, size of wound, need for addition of factors, etc.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As used herein the phrase “treatment regimen” refers to a treatment plan that specifies the type of treatment, dosage, schedule and/or duration of a treatment provided to a subject in need thereof (e.g., a subject diagnosed with a neural tissue pathology). The selected treatment regimen can be an aggressive one which is expected to result in the best clinical outcome (e.g., complete cure of the pathology) or a more moderate one which may relief symptoms of the pathology yet results in incomplete cure of the pathology. It will be appreciated that in certain cases the more aggressive treatment regimen may be associated with some discomfort to the subject or adverse side effects (e.g., a damage to healthy cells or tissue). The type of treatment can include a surgical intervention (e.g., removal of lesion, diseased cells, tissue, or organ), a cell replacement therapy, an administration of a therapeutic drug (e.g., receptor agonists, antagonists, hormones, chemotherapy agents) in a local or a systemic mode, an exposure to radiation therapy using an external source (e.g., external beam) and/or an internal source (e.g., brachytherapy) and/or any combination thereof. The dosage, schedule and duration of treatment can vary, depending on the severity of pathology and the selected type of treatment, and those of skills in the art are capable of adjusting the type of treatment with the dosage, schedule and duration of treatment.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Experimental Materials and Methods

### Preparation of Coral Skeleton Matrix

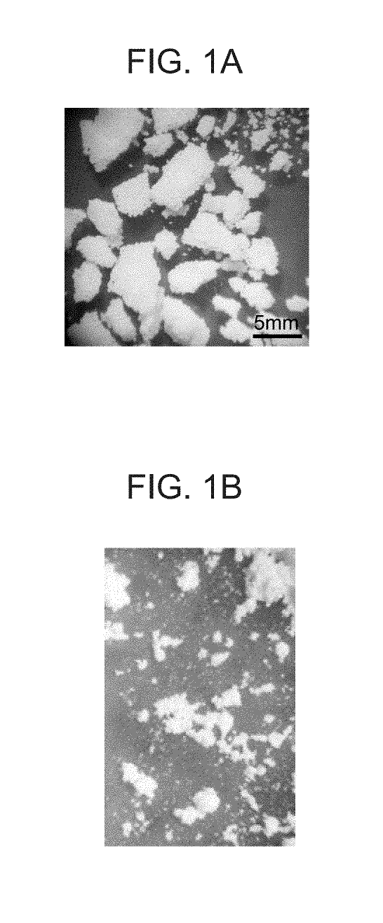

Biomaterial was derived from the exoskeleton of the coral Porites lutea. The skeletons were bleached with hypochlorite solution, rinsed with distilled water and dried in air. The organic residues within the skeleton were then removed with 10% NaOH solution for 5 minutes at room temperature. For further removal of organic deposits, the templates were washed with analytical H2O2 solution (30% by weight) for 5 minutes at room temperature. The templates were autoclaved and grained using a KometaBio grainer (Kometa, London, UK) (FIGS. 1A and 1B).

### Neural Tissue Culture

The culture method was based on a previously published procedure. Briefly stated, matrices and 12-mm glass coverslips were coated or not coated with poly-D-lysine (20 mg/mL). Next, hippocampi of postnatal rats were dissected and sectioned and placed on the coverslips, then covered with culture media. The culture medium was comprised of MEM containing 10% heat inactivated fetal bovine serum, 2 mM glutamine, and 0.76% glucose. On the following day, the medium was replaced with fresh MEM medium containing 0.30% glucose and B-27 supplements, and the cultures were maintained for few weeks at 37° C. in a humidified incubator with 10% carbon dioxide.

Preparation of Implants:

Collagen stock was prepared at a concentration of 4 mg/ml (in acetic acid) and titrated with NaOH to pH ˜7.4. Coral skeleton particles were then added to the gel to a concentration of 3%-6% coral exoskeleton (w/v). The composition was then buffered (final 1× Phosphate buffer saline (PBS)) and incubated at 37° C. for 30-60 minutes.

### Injury and Implantation

Motor cortex lesion is an experimental model of TBI. This type of brain damage causes motor impairments, providing appropriate conditions to test motor-recovery treatments. Briefly, mice (40-50 gr) were anesthetized using Ketamine 100 mg/Kg and Xylazine 10 mg/Kg and placed on a stereotactic apparatus. A 5 mm incision was made along the head and a 2.3 mm hole created in the skull at A/P (−2.3 mm), R/L 1.5 mm and U/D 1.5 mm coordinate, using a power drill. A 1.8 mm (diameter)×2.0 mm (depth)-sized wound was then created at the center of the hole by drilling into the cortex. The motor cortex lesion produces an injury that spans the thickness of the cortex as well as part of the hippocampus.

In some cases traumatic brain injury was simulated by blunt trauma directed to the cortical region of the brain, for example, by targeted injury with a heavy weight.

Following the injury, a 15 μl coral skeleton-collagen implant was inserted into the wound using forceps. The skin was replaced on the skull opening and closed with clips. The animals were supervised for about an hour after waking before being transferred back to their cages. The animals were weighed every day for the next 2 weeks.

### Immunofluorescence

Two weeks or one month post-implantation the animals were perfused with 4% paraformaldehyde (PFA) followed by overnight incubation with sucrose (4% w/v) in PFA 4% and 3 days with sucrose (30% w/v) in PFA 1%. Two coronal sections were made manually at opposite sides of the wound and the sections were fixed for 10 min at room temperature with 4% paraformaldehyde, exposed for 5 min to 2.5% Triton X-100, and incubated in PBS containing 3% goat serum overnight at 4 C.° with the following antibodies:

1. Monoclonal anti-dendritic protein microtubule-associated protein 2 (MAP2).

2. Polyclonal anti-glial fibrillary acidic protein (GFAP).

3. Monoclonal anti axonal marker Neurofilament M (NFM).

4. Monoclonal anti-nestin (neuroblasts).

5. Polyclonal anti-glutamate receptor 1 (GluR1).

6. Monoclonal anti-synaptic vesicle 2 (SV2).

Secondary antibodies were Alexa or CY3 conjugated goat anti-rabbit and goat anti-mouse. Cell nuclei were detected by 4′,6-diamidino-2-phenylindole (DAPI).

Immunofluorescent detection in tissue cultures at various stages of culture was performed using similar procedure.

Cells culture or brain section slides were analysed using a Zeiss LSM700 confocal microscope. Sections of 6-30 μm thickness were acquired using apochromat ×10 objective, and 3D images were reconstructed using the NIH Figi software.

### Weight Registration and Behavioural Tests (Open Field)

Following implantation, animals were returned to their cages and weight was documented daily. In parallel, 12 days following implantation animals were placed in a close cage (field) and their motility was video monitored. The total walking distance, time spent in the cell's center and corners were calculated by the Etho Vision 7.1™ software.

### Toxicology

Implanted and control brains were fixed with 4% paraformaldehyde and embedded in paraffin on 0, 1, 4 or 30 days (e.g. one month) following implantation. Tissue was sectioned (20 um thick) and the sections analyzed for infection, bleeding, edema and existence of activated macrophages using hematoxylin eosin (H&E) staining.

### Statistics

For statistical analysis of behavioral tests the EthnoVision version 10 (Nolus, Netherlands) software was used. Significance test used was GraghPrad Prism 5.2.

### Results

### Example I: Coral Skeleton Particles Implanted In-Vivo Promote Only Partial Recovery from Cortical Lesions in Adult Mice

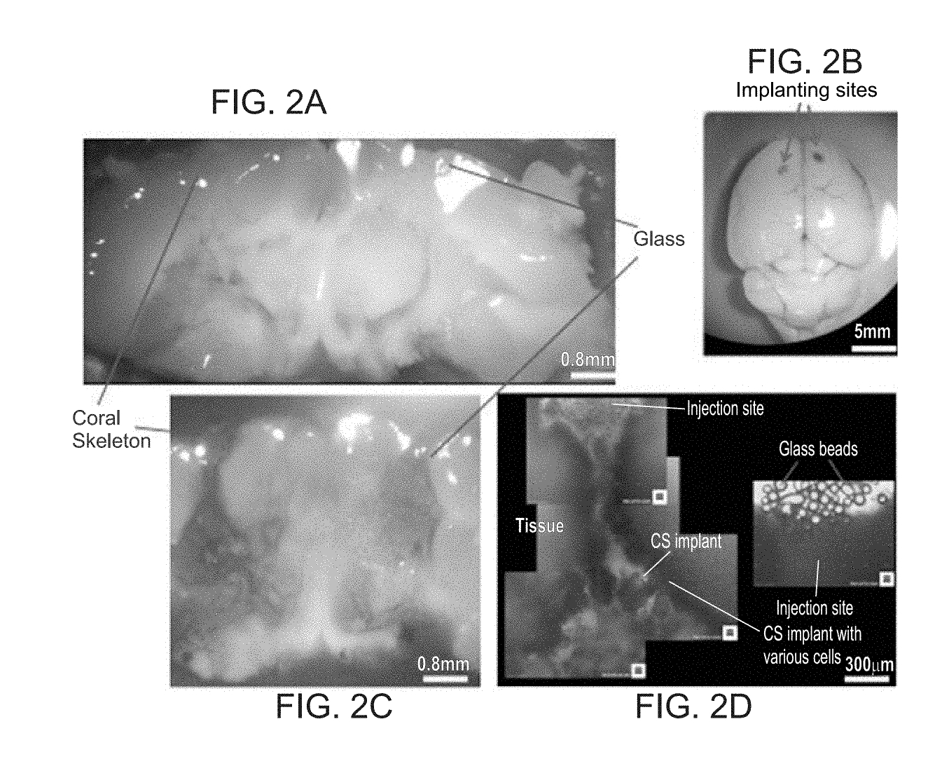

The regenerative capability of the coral skeleton as a graft was tested in vivo in cortical lesions of adult mouse brains. Large pieces (2-4 mm length) or particles of coral skeleton were inserted into 2-4 mm wide lesions in the middle to frontal cortex of 2-3 months old mice (40-50 gr), one in each hemisphere (FIG. 2B). See also methods). Glass beads (0.1 mm diameter) were used as a control implant and toluidine blue staining was used for general cell labeling (FIGS. 2C, 2D).

A few days following implantation, cell staining was significantly stronger around the coral skeleton implant than near the glass beads (FIG. 2C), suggesting a strong adhesion between cells in the wound and the skeleton. Moreover, whereas the coral skeleton particles were undisturbed by washing of the slide during the staining procedure, glass beads were dislodged and washed out (FIG. 2D) during the procedure, demonstrating the adhesive strength of the coral skeleton particles.

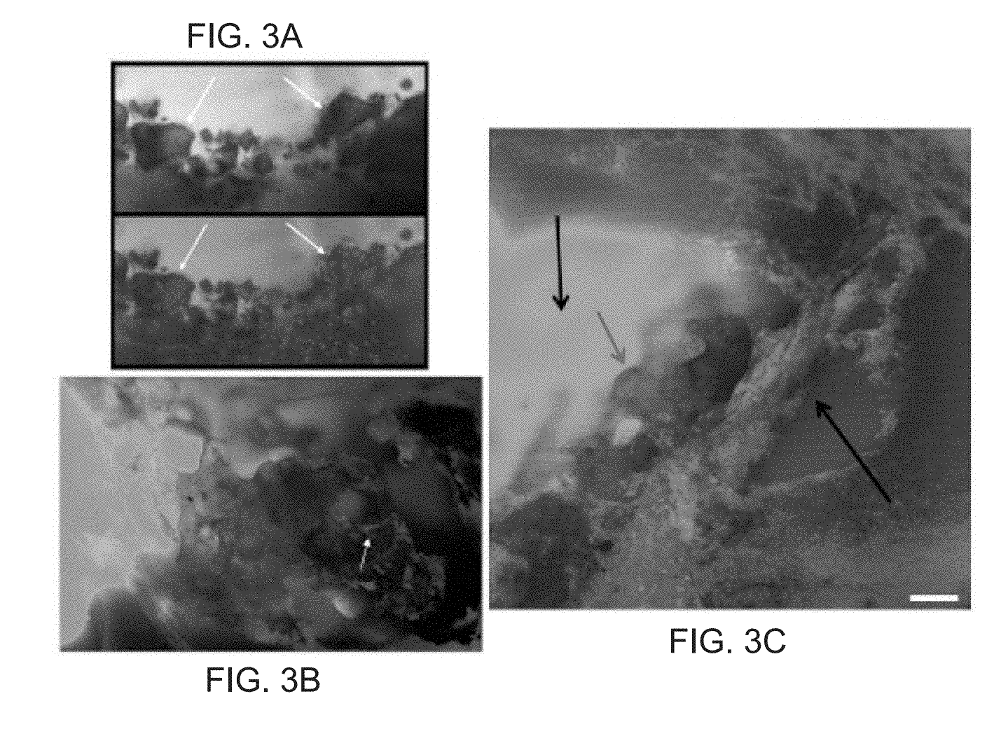

Further observation of cell distribution using DAPI staining confirmed that cells indeed adhere to the coral skeleton particles (FIG. 3A). Upon staining the coral skeleton implant was found to be associated with neurons (FIG. 3B) and astrocytes (FIG. 3C). Despite the fact that occasionally glial cells formed thick bridges across wounds, the newly generated tissue was mostly associated with the particles and occupied only a small portion of the wound. At one month after implantation, the number of cells found within the wounds ranged from several dozen to a few hundred.

### Biodegradability:

A significant reduction in the size of the coral skeleton implants, confirming the biodegradable nature of coral skeleton particles, was observed when implants were implanted in the brain. Typically, coral skeleton implants composed of small particles were extremely difficult to follow by one month after implantation.

### Example II: Collagen-Coral Skeleton Particles Implant Promotes Full Recovery with Neuron and Synapse Formation in Cortical Wounds

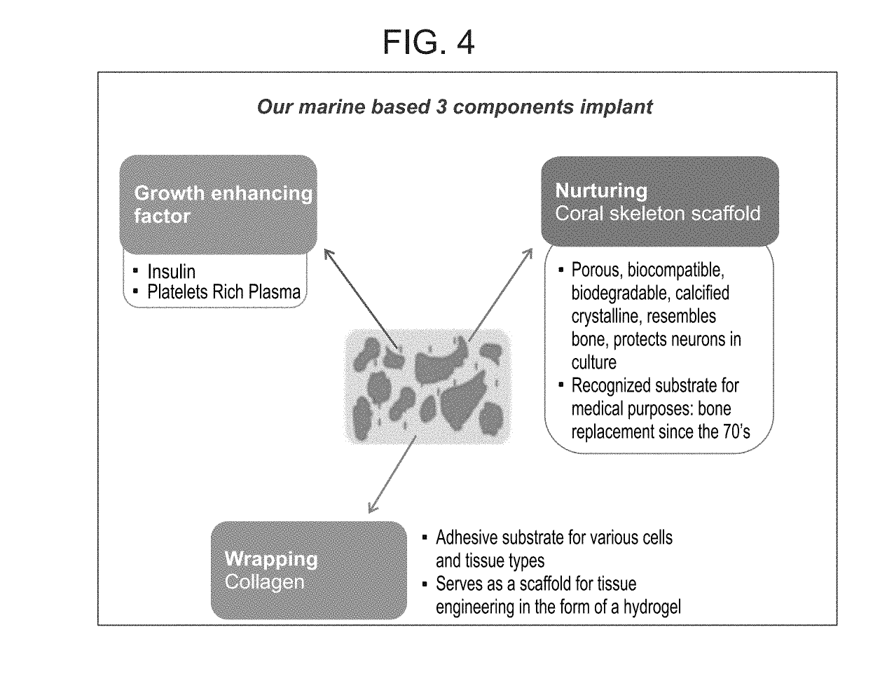

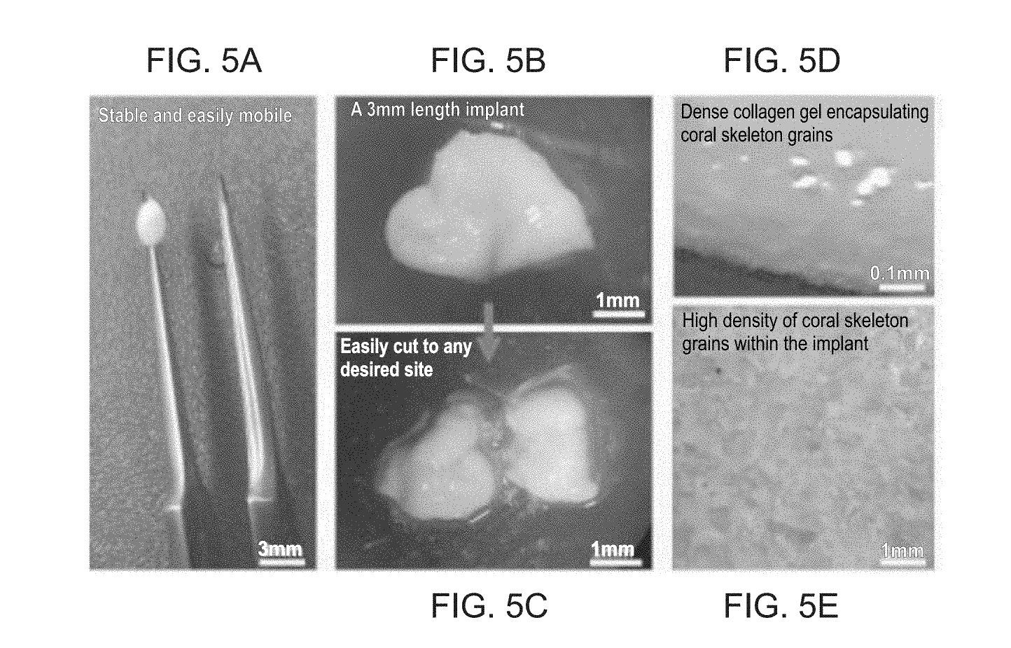

Addition of collagen to coral skeleton particles creates an implant having structural properties greatly differing from implants of coral skeleton alone. The coral skeleton-collagen implant is illustrated in FIG. 4. It is composed of coral skeleton particles of varying size (0.005-0.5 mm) encapsulated in a thin layer of collagen gel. The collagen can be free of, or supplemented with Insulin (12 μg/ml) or other additives (factors, antibiotics, drugs, platelets, etc). The result is an implant which can be easily handled, can comprise a high density of coral skeleton particles, is flexible and can be readily shaped for good fit into a wound (FIGS. 5A-E).

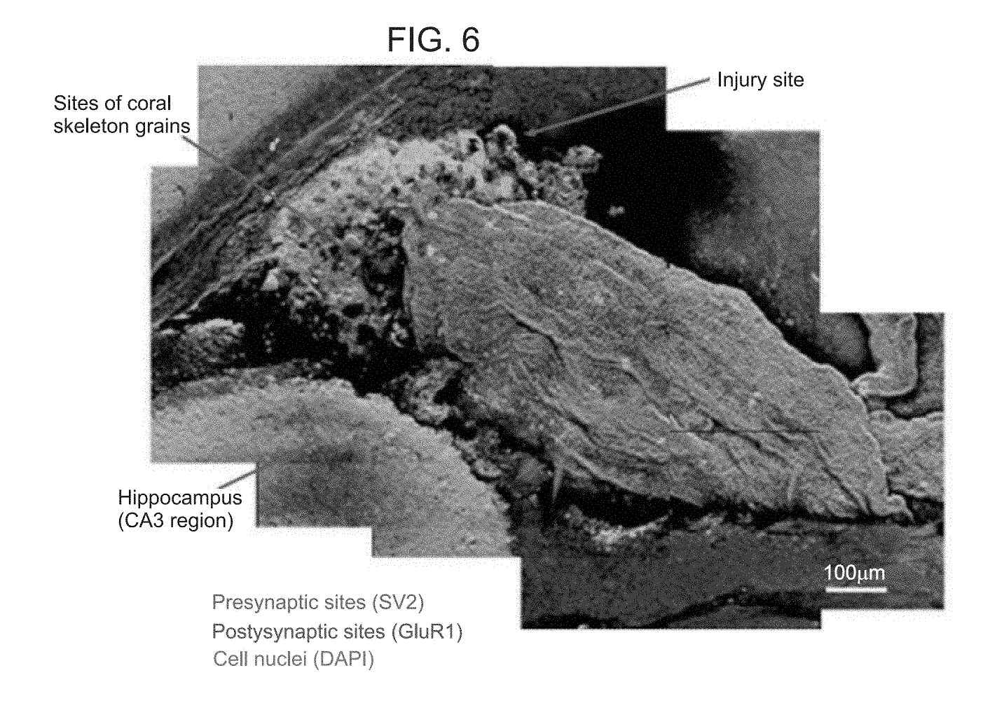

Encapsulation of coral skeleton particles in collagen gel caused a dramatic elevation of the capacity of the coral skeleton particles to regenerate nervous tissue. Inserting the collagen-coral skeleton implant into cortical injury sites in adult mouse brains caused accumulation of cells and presynaptic structures within the wound (FIG. 6). The properties of the tissue formed within the wound were quantified and compared to that of the hippocampal CA3 region and dentate gyrus region (DG, not shown) (non-injured). Cell density found at the wound was approximately 3×10⁵ cells/mm³, relatively high for wound healing, although about one order of magnitude lower than cell density at the CA3(arrow) and DG (not shown) regions.

Presynaptic terminals were evident as small (1-2 μm diameter) spots within the wound. These terminals expressed the synaptic vesicle 2 (SV2) protein to a higher extent than terminals located at non-injured hippocampi. By contrast, expression of the postsynaptic marker glutamate receptor R1 (GluR1) was lower within the wound than in the basal dendrites of neurons in the CA3 region of the hippocampus.

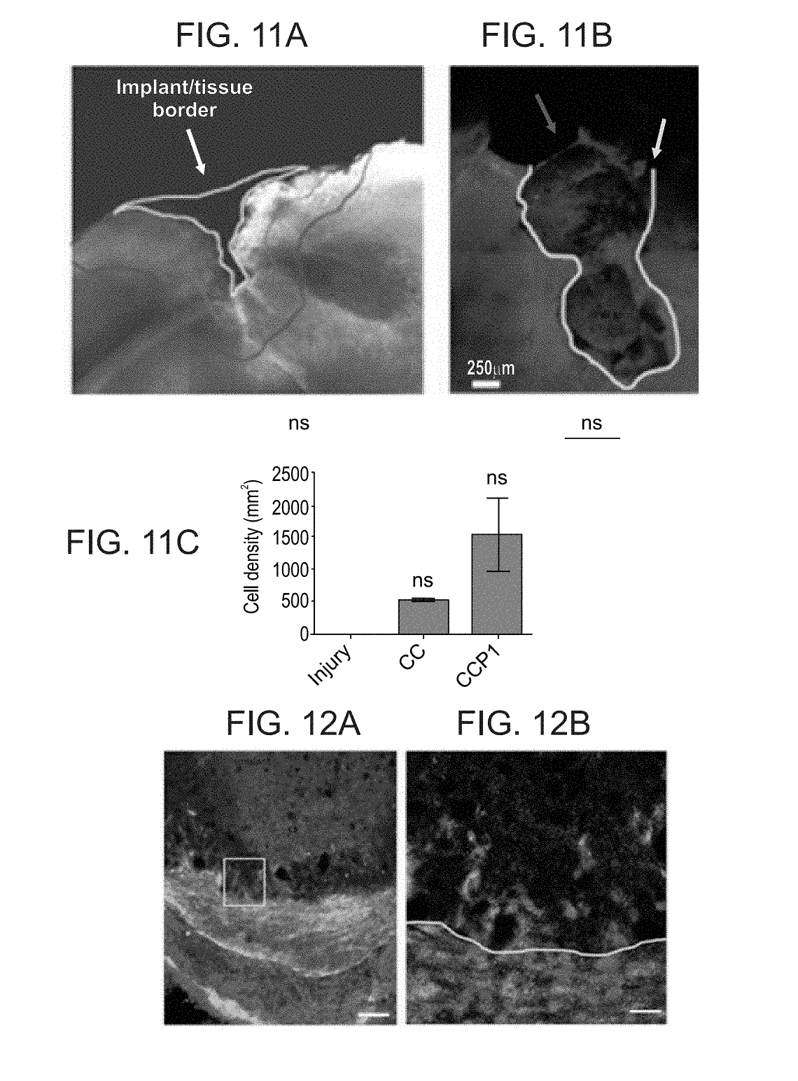

While untreated cortical wounds, lacking replenishment of neural cells in the damaged tissue, invariably collapse (FIG. 10A), cortical injury sites treated with coral skeleton-collagen implants are stabilized and maintain their structure due the implant and migration of neural cells (FIG. 10B). This is further borne out by the identification and quantification of neural cells migrating into the implanted wounds (FIGS. 11B and 11C (for quantification)), significantly abundant within one month (as shown here) of injury and coral skeleton implantation. It is important to note that while cell density within the wound is strongly affected by the presence of the implants of the invention (see FIG. 11C), measurement of the cell density in the tissue adjoining the wound cavity indicated that the wound surfaces are not significantly affected (data not shown).

Collagen-Coral Skeleton Implants with Factors

### Insulin and EGF:

Collagen-coral skeleton implants with added factors that might aid both the rate and extent of wound recovery were investigated. The first additives chosen were Insulin+EGF, a combination of factors that were observed effective in nervous tissue generation in vitro.

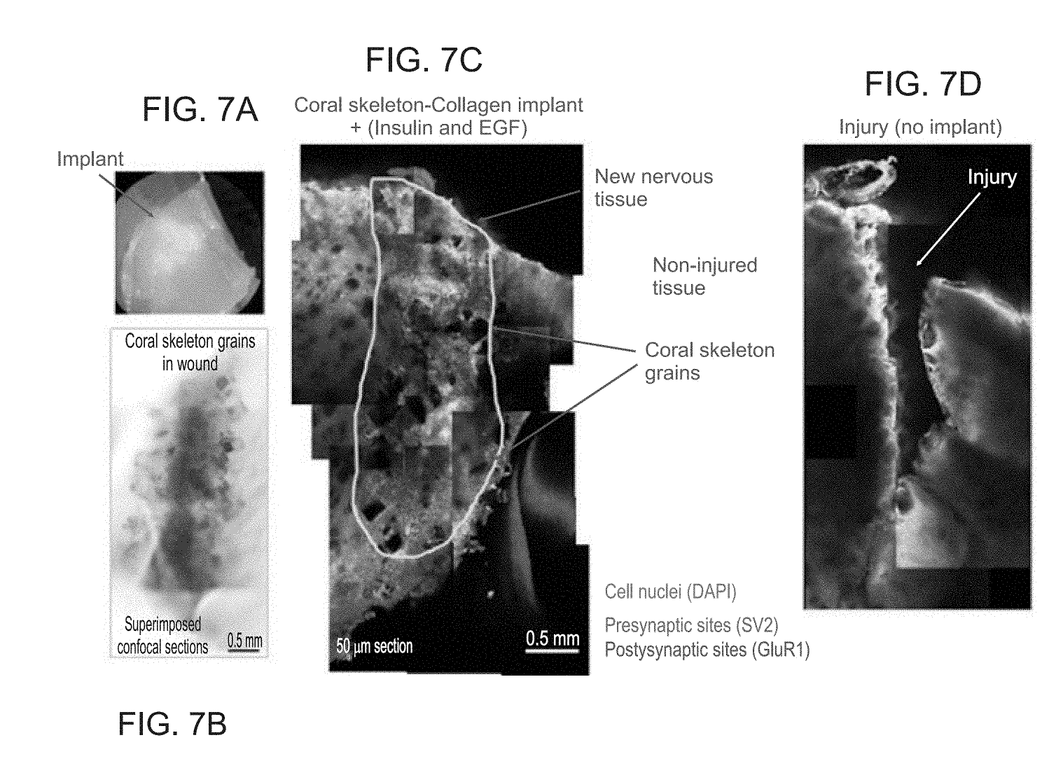

Implantation of the collagen-coral skeleton particles implant containing Insulin (12 ug/ml) and EGF (60 ng/ml) resulted in full recovery of the wound: By one month after implantation, the cortical wounds were filled with tissue, containing thousands of cells, with many prominent presynaptic—as well as post-synaptic sites (FIGS. 7A-D). No regeneration of neural tissue was observed in untreated, control (no implantation) wounds.

### Platelets:

In addition to their role in blood coagulation, platelets play a role in tissue regeneration. Preliminary results with platelets showed that in vitro interaction between coral skeleton particles and platelets cause platelet activation. Combining this information, we fabricated an implant composed of coral skeleton and collagen filled with platelets. To ensure activation of the platelets, adenosine-di-phosphate (ADP, 25 mM) was also added. FIGS. 8A and 8B show the cortical wound after implantation of collagen-coral skeleton particles implants comprising platelets and ADP, stained for astrocytes and neuroblasts (FIG. 8B). Astrocyte staining provides an indication of whether the implant is capable of breaking down or preventing formation of an astrocytic scar. FIG. 8B shows that, one month after implantation, the implant is not associated with a scar, and that the wound contained neuroblasts in a significant higher density than that found in non-injured areas. Accumulation of the neuroblasts in the wound suggests that the process of neurogenesis is active both nearby and within the graft.

Coral Skeleton-Collagen-Platelet-Rich Plasma+Insulin Implants

Implants comprising coral skeleton, collagen, platelet-rich plasma (10,000-50,000 cells per implant—15 μl) and insulin (12 μg/ml) were prepared, and tested to assess their therapeutic effect on brain trauma, particularly traumatic brain injury. Cortical wound healing following implantation of coral skeleton-collagen-platelet-rich plasma+insulin implants indicated that the addition of insulin and platelets (e.g. platelet-rich plasma) to the implants strongly enhances both the magnitude and the character of their therapeutic effects on the injured brain tissue.

Methods:

Isolation of Platelet Rich Plasma (PRP):

Sabra mice were sacrificed using CO₂ and 1-2 ml of blood was recovered from each heart. The blood was mixed with citrate in a ratio of 9:1 and then diluted with PBSX1 in a ratio of 1:3, respectively. The tube was centrifuged at 400 g for 20 minutes at 4° C. The supernatant containing platelets and other plasma proteins was transferred into a clean tube and centrifuged again under the same conditions to remove remaining blood cells. The supernatant was transferred again into a clean tube and then centrifuged at 2,000 g for 20 minutes at 4° C. in order to precipitate the platelets. The supernatant was partially removed, leaving 1-2 ml of supernatant in the tube. The pellet was then suspended in the remaining plasma, constituting the platelet-rich-plasma (PRP).

Preparation of Collagen Hydrogel Implants:

Collagen type I from rat tail dissolved in acetic acid 0.02N to final concentration of 4 mg/ml was used for the preparation of the collagen hydrogel (Millipore). Collagen solution for polymerization was prepared according to Cultrex rat collagen I polymerization protocol (Trevigen, USA)(briefly, type I collagen is diluted to the desired concentration with phosphate buffered saline and sodium bicarbonate and gently mixed) to a final concentration of 1.5 mg/ml. Coral skeleton powder (0.9 mg/15 μl) and/or Insulin (12 μg/ml) and/or PRP (10,000-50,000 cells/15 μl) were added to the collagen solution. The solution was then dispensed into portions of 150 in a 96 wells plate and placed in 37° C. incubator for 30 minutes. The collagen implants were then preserved on ice until the implantation process.

Results:

When the migration of neural cells into a cortical wound following implantation of coral skeleton-collagen (“CC”) or coral-skeleton-collagen-platelet-rich plasma+insulin (“CCPI”) implants is visualized (for example, FIG. 11B), the regenerative effect of the implant is immediately evident, compared to the control, unimplanted wound site (FIG. 11A). Quantification of the migration of neural cells, however, showed a significant increase in wound-site cellular density (FIG. 11C) with CCPI implants, compared to that observed with CC implants.

Further investigation revealed that the cells migrating into the cortical wound site following CCPI implantation, and replenishing the wounded tissue, comprise a significant portion of cells characterized by neural-cell specific markers (FIGS. 12A and 12B, green=nestin), indicating migration of neuroblasts.

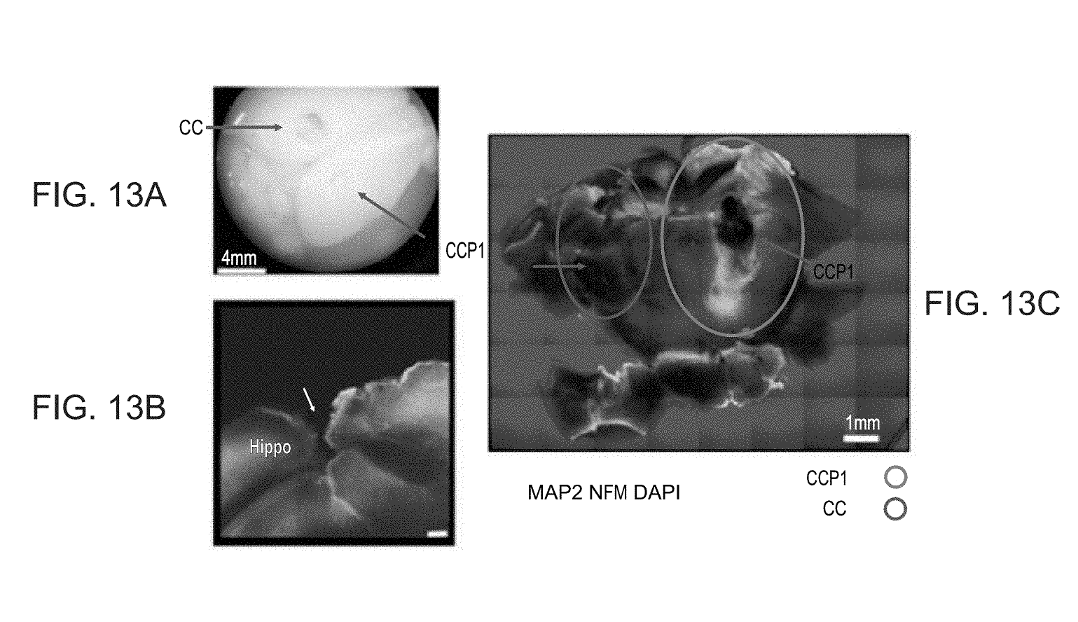

Gross morphological (whole brains) comparison of traumatic brain injury sites treated with CC or CCPI implants indicated more extensive wound repair with CCPI, compared with CC implants (FIG. 13A). This was clearly reflected in the character of the regenerating tissue: after one month, density of axons and dendrites (neuronal-specific markers MAP2, NFM) in the site of the wound treated with the CCPI implants was significantly higher than in wounds implanted with CC implants (FIG. 13C). In the absence of implants, no clear layers of axons or dendrites were observed (FIG. 13B).

Taken together, these results indicate that implants comprising coral skeleton-collagen and implants comprising coral skeleton-collagen-platelet-rich plasma+insulin not only prevent scarring but are highly effective in promoting repair and regeneration of neural tissue in brain injuries, as exemplified in the recovery of neural tissue following cortical wounds. Yet further, the coral skeleton-collagen-platelet-rich plasma+insulin implants appear to be more effective in promoting this repair and regeneration than the coral skeleton-collagen implants.

### Example III: Safety and Toxicity: Coral Skeleton Implants are Safe, and do not Alter Animal Mass or Behaviour

Safety and toxicity of the implants were assessed via tissue pathology as well as via physiological parameters and behavioral performance of the whole animal. Experiments were conducted to assess whether the implants caused damage such as inflammation, edema, bleeding and leukocyte accumulation in the subject. For pathology, control and implanted brains were processed, paraffin embedded, sectioned and stained with H&E. Both brains of animals receiving coral skeleton implants without additives, and brains of animals receiving collagen-coral skeleton implants exhibited no signs of toxicity compared with brains of control animals that were injured without receiving implants.

### Animal Mass and Behaviour

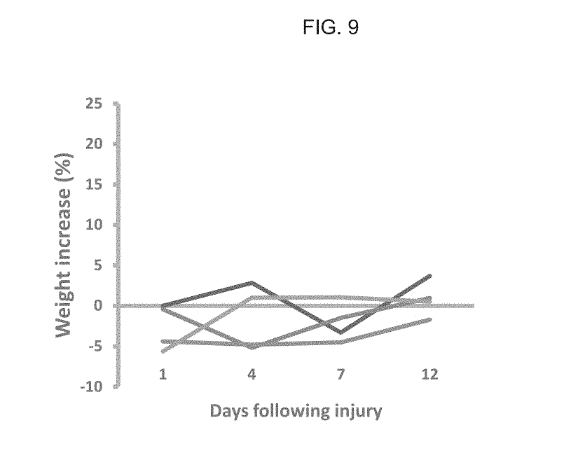

Grafting implants of coral skeleton alone or collagen-coral skeleton implants did not cause any significant change in animal mass compared to that of animals with cortical injury but without no implant, up to one month from the onset of the experiment (FIG. 9).

Similarly, the coral skeleton implants did not influence the animal's behaviour, as verified through three types of open field tests: ‘Total distance travelled’; ‘Average velocity’ and ‘Total time in peripheral zone’. All these tests are linearly correlated measures of anxiety.

Comparison of the results showed that animals that underwent injury with or without implantation had similar elevation of anxiety, compared to the non-injured animals (results not shown).

### Example IV: Coral Skeleton-Collagen Implants with Additives Induce Recovery of Functional Deficits Following Traumatic Brain Injury

Cortical injury (traumatic brain injury from blunt trauma) causes discernible motor and behavioral impairment. When motor and behavioral function of animals following such cortical injury was assessed in open field tests, improved recovery in parameters of motor performance (elevated walking distance and velocity, FIG. 14A), anxiety (increased center entries, FIG. 14B) and curiosity (rearing behavior, FIG. 14C) at one month was observed in mice implanted with coral skeleton-collagen-platelet-rich-plasma+insulin implants following the traumatic brain injury (FIGS. 14A-14C, black columns), compared to their non-implanted controls (FIGS. 14A-14C, blank columns).

Taken together, the results presented herewith provide strong evidence for therapeutic applications of coral skeleton implants, and in particular coral skeleton implants with additives such as coral skeleton-collagen (“CC”) or coral-skeleton-collagen-platelet-rich plasma+insulin (“CCPI”) implants, in treatment of neural tissue injury, including but not limited to traumatic brain injury.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method for regenerating or repair of neural tissue comprising contacting the neural tissue with a composition comprising porous crystalline calcium carbonate or calcium phosphate particles and a biocompatible polymer.

2. The method of claim 1, wherein said neural tissue comprises a tissue selected from the group consisting of neuronal tissue, glial tissue and central nervous system neural tissue.

3. The method of claim 1, wherein said neural tissue comprises central nervous system tissue.

4. The method of claim 3, wherein said neural tissue comprises brain tissue.

5. The method of claim 1, further comprising contacting said neural tissue with a nerve growth or regeneration agent.

6. The method of claim 5, wherein said nerve growth or regeneration agent is selected from the group consisting of insulin, epidermal growth factor (EGF), brain derived neurotrophic factor (BDNF), glial cell-derived neurotrophic factor (GDNF), nerve growth factor (NGF), VGF nerve growth factor (VGF), neurotropin-3 (NT3) and cytokines.

7. The method of claim 5, wherein said composition comprises said nerve growth or regeneration agent.

8. The method of claim 1, wherein said contacting neural tissue is effected in vivo in a subject in need thereof.

9. The method of claim 8, wherein said subject is a human subject.

10. The method of claim 1, wherein said neural tissue is injured or damaged neural tissue.

11-12. (canceled)

13. The method of claim 1, wherein said composition comprises cells and, optionally, wherein said cells are seeded upon said composition.

14. (canceled)

15. A composition for regenerating or repair of neural tissue comprising porous crystalline calcium carbonate or calcium phosphate particles, a biocompatible polymer and a nerve growth or regeneration agent.

16. (canceled)

17. The composition of claim 15, wherein said biocompatible polymer is biodegradable and, optionally, wherein said composition is biodegradable.

18. (canceled)

19. The composition of claim 15, wherein said porous calcium phosphate particles comprise hydroxyapatite particles and said porous crystalline calcium carbonate comprises aragonite.

20-21. (canceled)

22. The composition of claim 15, wherein said porous crystalline calcium carbonate comprises acellular coral exoskeleton and optionally wherein said porous crystalline calcium carbonate comprises a Porites lutea coral exoskeleton.

23-25. (canceled)

26. The composition of claim 15, wherein said biocompatible polymer comprises a natural polymer selected from the group consisting of collagen, albumin, fibrinogen, elastin, silk, hyaluronic acid and chitosan.

27-30. (canceled)

31. The composition of claim 15, wherein said polymer comprises 2-6 mg/ml collagen.

32-34. (canceled)

35. The composition of claim 15, wherein said composition comprises said porous crystalline calcium carbonate particles and said biocompatible polymer in a ratio of at least 33 μg calcium carbonate particles per milliliter biocompatible polymer.

36. The composition of claim 15, wherein said composition comprises at least one of insulin, EGF and platelets, and, optionally, wherein said platelets comprise platelet-rich-plasma.

37-38. (canceled)

39. The composition of claim 15, wherein said composition comprises cells.

40. A pharmaceutical composition comprising the composition of claim 15 and a pharmaceutically acceptable carrier.
